SEQUENCE LISTING (14 sequences)

SEQ ID NO: 1 - DNA, 24 bp, Artificial Sequence
  Synthetic
ggacgtatccacctcaggtacaca

SEQ ID NO: 2 - DNA, 27 bp, Artificial Sequence
  Synthetic
cgtgtttcacaaactgcaccagtacca

SEQ ID NO: 3 - DNA, 20 bp, Artificial Sequence
  Synthetic
aagtacccctatcgcgtgtg

SEQ ID NO: 4 - DNA, 20 bp, Artificial Sequence
  Synthetic
aaaagagaccgcgtctctgg

SEQ ID NO: 5 - DNA, 28 bp, Artificial Sequence
  Synthetic
cgctctttttgtcagtctgaatcgaccc

SEQ ID NO: 6 - protein, 247 aa, Human cytomegalovirus
MILWSPSTCSFFWHWCLIAVSVLSSRSKESLRLSWSSDESSASSSSRICPLSNSKSVRLPQYPRGFGDVSGYRVSSSVSECYVQHGVLVAAWLVRGNFSDTAPRAYGTWGNERSATHFKVGAPQLENDGALRYETELPQVDARLSYVMLTVYPCSACNRSVLHCRPASRLPWLPLRVTPSDLERLFAERRYLTFLYVVLVQFVKHVALFSFGVQVACCVYLRWIRPWVRGRHRATGRTSREEEAKDD

SEQ ID NO: 7 - protein, 907 aa, Human cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 8 - DNA, 4895 bp, Human cytomegalovirus
ggcgcagtccgcggcagggttccggccgtgctgcggtccgcacgctgcgcccgctcccgcctgcctcccgccctaccccccaccctccccggccgaggcccggcgctggtccgtccgcgggcccgtcccaccgccctggagcaccatccggggccgtgggccgggcaccgggcgcggcccgctccggacctcggccgggggtccctcccctccccccgctcgaccccccatccgacggcccggccgggctgggacccccgcaccggggtcccggttcccgtccgcggcccggggggacccgagcgggggcttcccacccccaccccgctcctccccgggctccggcccgggatccctcgctgctcccggcgacctccgccggcttcccggtccacccgccgcggaacggacgggacccggggtccgcgcccttcccctccccccacggggggctgggtcgcggaccccggttcctaggctcgttccgcggtgggcgaccggggatcccccacccagctccccttcccggcccgccccgctggcttttgggccccctccgggcttttttccggctgggggtcgcggcggtcggccgacgacgacggtaggtgggccgggtggacggtggtggggacgggcgacgccccggctcgacggcagtcggtcccggaaggttgggggctgggggcccggtcaggagctccgggagcggggtcgaccgcgacggcttccgggtctcgcgacggctccctctcggcggctccggttgggctctcctcccccttctcgagggtccggccgccagtcgtgaccgggggtccctcggcctagccgccggctttcgggctgccttatcctgggcgttggccggtcccgtgacgctcccttcccccactgctccccaaaaaagtccgcccgaaccgtcgcggcttgctggccctgggcgtggtcccccactcccctccccccatcggccgcccagccggggtcggcgcctcggaccccaccaggctgtggcgtgtgtgctggccgatgcggcggcgaggttgggtgtggccggaagcgctcggggtcgacggtgggccgccatgacacctcaattgtcgtcagtacgcccctccacaatcaccgtccccacacgatgggcccggcaggtcacccaacgttggttcaggcccagtcgggtttttccccggcacgaacgcacgtccccgtgggctccacgcgttttccaccctttcctggaggggtccggaacaccgtgaatccacggggagggtcccggcacgggccgaggagaccacgaccgtcccacccggcgtgtcgacccgtccgagacccgggaagggaacaggccccaccattttttttttcccttctccgatttgccgtggaaaacccgtgaaccgatacgggtacagacggccgaaaaaaattcgagacaatacgacggcagggcgtgattttctcccccatccgacaaaaccgtgtcccttaaaattcctcacctttctctgttcaaatggccccgaaactgtaaaacaccgtttgaccgcacctcaaccggcgccatcttggtgacctcgacggttctctcgctcgtcatgccgttctgagctccgccatggcggacgagagaaaatggcgtcgagagcctaggagcgttttcgctccaggtgggtaaaaaaatagcacgataacttttctgtgctttttttgagacgttttagaagagcttttttctgctcagagcgaaaaaatgatagccctgaaaatctcgacgagtctggccgagcggcgccatcttggaggaggggcgagtcgcgggcaccgcctcggtaccccctggccgaggcgagtccgcggtcgccgcctgttccgtgatgctacctagagggcgctgtcgaggcgactcttcctgttttcgccctgagggctaacggtcgctgacgtcaaaccatctcgtgctcgctgagtcacatccggttgttgacaagcgatggaggaccgcacccaaagtgcgccctctagtcatcgcgcctgaccccttttataaactgctcgaagaaaagaacaccttatgtgaaaaaatacagaatgatgacaagttcatccaacacaaccgctcaacaacgccatatctatcagtgtccaaaaactatcttctatcctttgaaactataaatgctgcctatatacatatttagtatccaagactcttaccacgtagacgaaaagaagtgatacaatgatcttgacgtgtatcgtctatatcgtgctagatatattcagataagacgcgcaaaccatagatttctcatcagtatcatgaaagacctatagctctatatacgaacctagtcattttaggacagccgccggagaagccgacgagggatcgggcgggtgcagccagaacctcacgcccgatcccgcctccggtaggcgatttgcatctgtttggtaaaaagctcataagtctgtatgtgacctatatatatattatacgctatgtacaccgaactgtcgctgttgtataagaagaaaaaactctccatatttatatcgtctgaatttttgcttgatagacacgtgtttggaactctgtccccccacgttttcactgtgtataacaaaaatatgtgtttctcaaaagatcttgaggtgtttgaaaacgggggaaacctgcgtttgggtgcgctaagccccggactgggacgtagccggcgtccggcacctatatttttctatttttttttacaaaatatatgatgaaccaagaataaaactctagctctcgtctatttttaatatgctctacttagaacctttttaatgacagaatgaactccatgttatacgctctttatatagtttctctgcactaacctttaaaaccgtatccttccctgttgtacaaatcatcttttgatacacaatgatgacctgatatccctccatatatatgatcggatattattccgttagacttgtcctcctttttttttcctcatctcctgtatctggagatatatgttgaccaccaccgccatgaccaccaaaaagctagccgtcacgactagaaatgtgtaggattcggactttccgttcgagaagaaagagaccgcgtctctggacgctctttttgtcggtctgaatcgacccgggatacgtaagagagcggccctacatcggggggcgctcgagaccgacgacgttccatctgaccagaaaaaaaaaaggcacccctcggtagcgacctctcaccatcgtttgcccgtccgcccgtcctccgtagccatcatcatcatcatctcaggctctatcggtaccatcgttgtcatctgaaaaaaaaactgcctcacccacctgcgtaaaaacaccatctttccggaggtgcggtaagacgggcaaatacggtcgtgccgaggcaaaaaaaaatgcaccatcgacaccacaccctcatgagcaccacctgtcggtgttggtcgtcctccatcgttctctacgaacatctcgacgcccgggtgacggacgacggcaagacgtcccggagaagacggtgttctctcgggcggtacgctctctggatctataatatctatagtagctaaacgagactgtgagtacgacgaaccacatcatcttttttttatgttgcttctttagaaaatgacttatgtcgacgacactcggcatcagccatctcgtgaaacacgctcgcttttcgtctctccaaggaacactgggtccgctgaaagggaccgtgtaccgaccaaagcaaaaaacacacacgtagtaacatgatcaaccacgtctgaatgacacgaaaacacaatcgtataacgctctattcatggaacgaacttggaataaaaaaaccatcgcaggccagaggctaagccgaaaccgtccggggaagcgggcgcgagttttccgacttagtctctggtgctcgttgagcctcttttttttttttctgattctctgaagaatcaccgtcacagccctatgacgcgaaatcaattgctagaacataaacgttctcaacaggtatgaaatgaacaaactagatgatgctataaccttatattgtgtgtatatagataggtgtgaaatttgtaggataaaaagtgtcgttgtatgatgcacaacgatcgtgaaactggagactgtagctctctaccgaatgcaaatacacaaatgacatcgattcccgtccccacataaagaaatgtgctttactgtgaaagaatgaagaagattcttgttcctcgtacgacggggccctcgctcatcgtgcctcttcccccctccgggagaggggacgtcggggccctccgtcgcaccgggccgaagccagtgaaatgtttactacactgtcatcagaatatatgatgtatattatttcctccaaactcctcaccatagccaccaattcgcatcacttaagaaagtagtagcaaccgcggcggcggcgaccggccggtcgtcgtctcctcgtcctcaaatgttgtacatgtgcagaaaaatgtgtaaatacgtgttatttatcccatgcgtcttgtacatagatatatgtttttatatacgctatttatactttatatatccttttgcataaccatagacagtcaaggattttaatgatttgctcatccgcctttgagccatcgcttaggagttagttcctctatgttctcggcccaccttttcgactacagtagcaaacccttgtactaccaccccgataaaaaccacatcatcatcgtcaccacgacctggaaacgacacacgttctcccccaatcttgggcatgtgtatatatatataaagaatgggagggagaggacgtggggctcgagaagaaataaacgccaagctcgattcgaacc

SEQ ID NO: 9 - DNA, 744 bp, Human cytomegalovirus
atgatcctgtggtccccgtccacctgttccttcttctggcactggtgtctgatcgcagtaagtgtactctcgagccgctccaaggagtcgctccggttgtcgtggtccagcgacgagtcgtctgcgtcctcgtcgtctcgtatctgtccgttgtcaaatagcaagtccgtccgtctgccccagtaccctcgcgggtttggggacgtgtccggctaccgtgtctccagttccgtgtccgaatgttacgtccagcacggggtactcgtggccgcctggcttgtgcgtggcaacttctccgatacggccccgagagcctacggcacctggggtaacgagcggagcgcgacccatttcaaggtcggcgcaccccagctggaaaatgacggggctctacgttacgaaactgagctcccacaggtggacgcacgactctcttacgtgatgttaaccgtttacccctgcagtgcctgtaaccggagcgtgctccattgccggccggcgtccaggctgccctggttgccgctgcgggtaacgccctcagacttggaacggctgtttgcggagagacgctacctgacattcctctatgtggtactggtgcagtttgtgaaacacgtagccttgttcagcttcggtgtccaggtagcctgctgtgtgtacctgaggtggatacgtccctgggtccgaggtcggcaccgcgccaccggaaggacctcacgggaagaagaggctaaagacgattga

SEQ ID NO: 10 - DNA, 2724 bp, Human cytomegalovirus
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctatcagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctctgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcttgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtctga

SEQ ID NO: 11 - DNA, 23 bp, Artificial Sequence
  Synthetic; IEA1 Primer P1
caagcggcctctgataaccaagc

SEQ ID NO: 12 - DNA, 24 bp, Artificial Sequence
  Synthetic; IEA1 Primer P2
ctcttcctctggggcaacttcctc

SEQ ID NO: 13 - DNA, 23 bp, Artificial Sequence
  Synthetic; MIE Primer P1
gggtgctgtcctgctatgtctta

SEQ ID NO: 14 - DNA, 23 bp, Artificial Sequence
  Synthetic; MIE Primer P2
catcactctgctcactttcttcc

(19) United States
(12) Patent Application Publication
(10) Pub. No.: US 2021/0324488 A1
(43) Pub. Date: Oct. 21, 2021

(54) METHODS, KITS, AND PRODUCTS FOR DETECTION OF CYTOMEGALOVIRUS INFECTION

(71) Applicant: Gravid Therapies, Inc., San Francisco, CA (US)

(72) Inventor: Daniel La Caze, San Francisco, CA (US)

(21) Appl. No.: 17/104,656

(22) Filed: Nov. 25, 2020

Related U.S. Application Data
(63) Continuation of application No. PCT/US2019/035021, filed on May 31, 2019.
(60) Provisional application No. 62/679,651, filed on Jun. 1, 2018.

(51) Int. Cl. C12Q 1/70; G01N 33/569

(52) U.S. Cl. CPC C12Q 1/705; G01N 33/56983; C12Q 2600/106; G01N 2469/20; C12Q 2600/158; G01N 2333/045

(57) ABSTRACT

Methods of determining a presence of active primary cytomegalovirus (CMV) infection are described herein. The methods can include determining a presence or absence of anti-CMV IgG in a sample of a subject. The methods can include determining a presence or absence of CMV nucleic acids in a subsequent sample of the subject. Kits and product combinations useful for performing the methods are also described.

### REFERENCE TO RELATED APPLICATIONS

The present application is a Continuation Application of International Application No. PCT/US2019/035021, filed May 31, 2019, which claims the benefit of U.S. Provisional Application No. 62/679,651, filed Jun. 1, 2018, the contents of which are incorporated herein by reference in their entirety.

### REFERENCE TO SEQUENCE LISTING

The present application is being filed along with a Sequence Listing in electronic format. The Sequence Listing is provided as a file entitled GTIN001C1SEQLIST.txt, created and last saved on Nov. 24, 2020, which is 23,225 bytes in size. The information in the electronic format of the Sequence Listing is incorporated herein by reference in its entirety.

### FIELD

Some embodiments herein relate generally to molecular biology methods, kits, and product combinations for the detection of cytomegalovirus (CMV). Some embodiments relate to methods of treating CMV infection.

### BACKGROUND

Cytomegalovirus (CMV) infections are one of the leading causes of birth defects, developmental impairment and subsequent long-term sequelae. The most damage is caused to a fetus when the mother acquires a primary CMV infection during the earlier half of gestation. Since most infections are sub-clinical or mild for subjects, there are often no defining symptoms to indicate a CMV infection over another type of infection that may provide no impact to the fetus.

### SUMMARY

In some embodiments, a method of determining a presence of active primary cytomegalovirus (CMV) infection is described. The method can comprise receiving a sample obtained from a subject at a first stage. The method can comprise determining a presence or absence of anti-CMV IgG in the sample. The determining can comprise an immunoassay test, in which an absence of anti-CMV IgG indicates that the subject is at-risk for CMV infection such as active primary CMV infection (for example at-risk of developing or having a fetal-damaging CMV infection). If the subject is at-risk, the method can comprise monitoring a subsequent sample of the subject obtained from the subject at a subsequent stage. The subsequent sample can comprise nucleic acid of the subject, and the monitoring can comprise detecting a presence or absence of CMV nucleic acid sequences in the subsequent sample by a high sensitivity test. An absence of anti-CMV IgG in the sample and a presence of CMV nucleic acid in the subsequent sample indicate a presence of active primary CMV infection. The subsequent sample can be from an at-home sample collection as described herein. In some embodiments, a method of determining a presence of active primary CMV infection is described. The method can comprise collecting a sample from a subject at a first stage. The method can comprise receiving a determination of a presence or absence of anti-CMV IgG in the sample. The determination can comprise an immunoassay test, in which an absence of anti-CMV IgG indicates that the subject is at-risk for CMV infection such as active primary CMV infection (for example, at-risk of developing or having a fetal-damaging CMV infection). If the subject is at-risk, the method can comprise receiving results of monitoring a subsequent sample of the subject obtained from the subject at a subsequent stage, the subsequent sample comprising nucleic acid of the subject. The monitoring can comprise detecting a presence or absence of CMV nucleic acid sequences in the subsequent sample by a high-sensitivity test. An absence of anti-CMV IgG in the sample and a presence of CMV nucleic acid in the subsequent sample indicate a presence of active primary CMV infection. In some embodiments, a method of determining a presence of active primary cytomegalovirus (CMV) infection is described. The method can comprise screening a subject as having an immunodeficiency, wherein the immunodeficiency indicates that the subject is at-risk for CMV infection. The method can further comprise monitoring a subsequent sample of the subject obtained from the subject at a subsequent stage, in which the subsequent sample comprises nucleic acid of the subject. The monitoring can comprise detecting a presence or absence of CMV nucleic acid sequences in the subsequent sample by a high sensitivity test. A presence of CMV nucleic acid in the subsequent sample can indicate a presence of active primary CMV infection. In some embodiments, the immunodeficiency comprises human immunodeficiency virus (HIV) infection or treatment with immunosuppressants. In some embodiments, for any method described herein, the method further comprises screening the subject as having an occupational risk of CMV infection prior to determining the presence or absence of anti-CMV IgG. In some embodiments, for any method described herein, the subject is screened as having an occupational risk of CMV infection if the subject is a nurse, elementary or middle school teacher, nursing or psychiatric or home health aide, child care worker, teacher assistant, primary child care provider for a preschooler (such as a parent or other caregiver who has one or more children under the age of five who are in preschool or day care), and/or is exposed to one or more children under the age of five multiple times per week. It is noted that a subject may have an “occupational risk” if she has other young children (especially under the age of 5) in daycare or otherwise exposed to young children, even if this exposure to young children is not a vocation or livelihood. In some embodiments, for any method described herein, the method further comprises recommending the subject for receiving CMV therapy if the monitoring detects a presence of CMV nucleic acids in the subsequent sample. In some embodiments, for any method described herein, the CMV therapy comprises an antiviral compound selected from the group consisting of: acyclovir, valacyclovir, ganciclovir, valganciclovir, foscarnet, cytogam and cidofovir. In some embodiments, the CMV therapy comprises an antiviral compound selected from the group consisting of: valacyclovir, ganciclovir, valganciclovir, foscarnet, and cidofovir. In some embodiments, the method further comprises the CMV therapy comprises valacyclovir. In some embodiments, the method further comprises monitoring subsequent samples of the subject after the start of the CMV therapy to detect changes in viral load or titer. A decrease in viral load or titer can indicate a response to the CMV therapy. The CMV therapy may be continued until there are two sequential negative high-sensitivity CMV tests. In some embodiments, for any method described herein, the subject is pregnant and/or a transplant subject. In some embodiments, for any method described herein, the subject is pregnant, and the therapy is recommended within three weeks of detection of infection, during the first half of the pregnancy of the subject, and/or prior to transmission to a fetus or appearance of symptoms in a fetus. In some embodiments, for any method described herein, the subject is pregnant, and the therapy is recommended for administration in the first trimester of pregnancy or the first half of the second trimester of pregnancy. In some embodiments, for any method described herein, the CMV therapy is selected from the group consisting of: an antiviral compound, a biologic, an immunotherapy, and termination of a pregnancy, or a combination of two or more of the listed items. In some embodiments, for any method described herein, the CMV therapy comprises an antiviral compound at a prophylactic dose that is lower than a lowest dose of the antiviral compound approved by a regulatory authority (e.g., the FDA or EMA) for CMV viremia. In some embodiments, for any method described herein, a presence of anti-CMV IgG is determined, and the method further comprises determining an avidity of the anti-CMV IgG for CMV. If the anti-CMV IgG has a low avidity for CMV, the method can further comprise monitoring the monitoring the subsequent sample of the subject obtained from the subject at a subsequent stage. In some embodiments, for any method described herein, the subsequent sample comprises urine or saliva. In some embodiments, for any method described herein, the subsequent sample comprises stabilized, preserved CMV nucleic acid. In some embodiments, for any method described herein, the subsequent sample was collected from the subject in the absence of any phlebotomist, nurse, physician or other licensed health care professional. In some embodiments, for any method described herein, the monitoring comprises, over a period of at least three weeks, detecting a presence or absence of CMV nucleic acid in at least three different samples, wherein each of the at least three different samples were obtained from the subject at different timepoints over the at least three weeks. In some embodiments, for any method described herein, monitoring the subsequent sample comprises high sensitivity tests on samples that were obtained from the subject at least one, two, or three weeks apart from each other. In some embodiments, for any method described herein, the sample comprises serum. In some embodiments, for any method described herein, the sample comprises plasma. In some embodiments, for any method described herein, the subject is at-risk for CMV infection. In some embodiments, for any method described herein, the method further comprises confirmatory testing if the absence of anti-CMV IgG in the sample and the presence of CMV nucleic acid in the subsequent sample are detected. In some embodiments, for any method described herein, the confirmatory testing comprises a confirmatory immunoassay test for anti-CMV IgG and/or IgM, and/or a confirmatory high-sensitivity test for CMV nucleic acid in the subsequent sample or another sample. In some embodiments, for any method described herein, the confirmatory testing comprises detecting consecutive increases in quantitative CMV nucleic acid measurements or in CMV viral titer measurements. In some embodiments, for any method described herein, the confirmatory testing comprises a confirmatory immunoassay test to confirm a presence or absence of anti-CMV IgG and/or anti-CMV IgM in the subsequent sample or another sample. In some embodiments, for any method described herein, the confirmatory immunoassay test indicates the presence of both anti-CMV IgG and anti-CMV IgM, then the confirmatory testing further comprises an avidity test to determine IgG avidity for CMV. In some embodiments, for any method described herein, the method further comprises collecting the first sample from the subject. In some embodiments, for any method described herein, the method further comprises providing a sample receptacle to the subject prior to receiving the subsequent sample. In some embodiments, for any method described herein, the method further comprises receiving the sample receptacle comprising the subsequent sample from the first subject by carrier. In some embodiments, for any method described herein, the method further comprises recommending to the subject that the sample be collected in a clinical setting and that the subsequent sample be collected in the absence of any phlebotomist, nurse, physician or other licensed health care professional. In some embodiments, for any method described herein, the subsequent sample was collected in a non-clinical setting in the absence of any of the phlebotomist, nurse, physician or other licensed health care professional. In some embodiments, for any method described herein, the method further comprises determining a presence or absence of anti-CMV IgM in the sample obtained from the subject at the first stage, in which, if there is a presence of anti-CMV IgG and a presence of anti-CMV IgM in the sample, the method further comprises determining an avidity of the anti-CMV IgG for CMV, and in which the anti-CMV IgG having a comparable or higher avidity to CMV than IgG of a CMV-developed control indicates that the subject has an active primary CMV infection. In some embodiments, for any method described herein, the method further comprises determining a presence or absence of anti-CMV IgM in the sample obtained from the subject at the first stage. If there is an absence of anti-CMV IgG and a presence of anti-CMV IgM in the sample, a near-term active primary CMV infection is determined, the method further comprises said monitoring the subject. In some embodiments, for any method described herein, the immunoassay test is selected from the group consisting of: immunoprecipitation, particle immunoassays, immunonephelometry, radioimmunoassay, enzyme immunoassay, fluorescent immunoassay, and chemiluminescent immunoassay. In some embodiments, for any method described herein, the enzyme immunoassay comprises an enzyme-linked immunosorbent assay (ELISA). In some embodiments, for any method described herein, the high-sensitivity test is selected from the group consisting of polymerase chain reaction, sequencing, rolling circle amplification, and isothermal amplification (such as multiple displacement amplification (MDA)). In some embodiments, for any method described herein, the polymerase chain reaction comprises reverse transcription polymerase chain reaction (RT-PCR). By way of example, the subject can be a pregnant female.

In some embodiments, a method of treating cytomegalovirus (CMV) infection in a subject is described. The method can comprise receiving a result of an immunoassay test to determine a presence or absence of anti-CMV IgG in a sample obtained from the subject. If anti-CMV IgG is absent from the sample, the method can comprise determining the subject to be at-risk for an active primary CMV infection. The method can comprise receiving results of a high sensitivity test to determine a presence or absence of CMV nucleic acid sequences in a subsequent sample of the subject. The method can comprise determining the subject to have an active primary CMV infection when anti-CMV IgG is absent and CMV nucleic acid is present. The method can comprise administering a CMV therapy to the subject determined to have the active, primary CMV infection. In some embodiments of the method, the presence of CMV nucleic acid sequences is determined in the sample, and thus, the subject is determined to have the active primary CMV infection. In some embodiments, for any method described herein, the method further comprises screening the subject as having an occupational risk of CMV infection prior to determining the presence or absence of anti-CMV IgG. In some embodiments, for any method described herein, the subject is screened as having the occupational risk of CMV infection if the subject is a nurse, elementary or middle school teacher, nursing or psychiatric or home health aide, child care worker, teacher assistant, primary child care provider for a child in preschool or daycare (such as a parent or other caregiver who has one or more children under the age of five who are in preschool or daycare), and/or is exposed to one or more children under the age of five multiple times per week. In some embodiments, for any method described herein, the screening is performed prior to receiving the result of the immunoassay test. In some embodiments, for any method described herein, the subsequent sample was collected from the subject in the absence of any phlebotomist, nurse, physician or other licensed health care professional. In some embodiments, for any method described herein, the subsequent sample of the subject comprises stabilized, preserved CMV nucleic acid. In some embodiments, for any method described herein receiving the results of the high sensitivity test comprises receiving the results of monitoring the subject, the monitoring comprising detecting a presence or absence of CMV nucleic acid in at least three different samples, wherein each of the at least three different samples were obtained from the subject at different timepoints over at least three weeks. In some embodiments, for any method described herein, determining the subject to have an active, primary CMV infection comprises receiving at least one of: a measurement of total CMV nucleic acid in the subsequent sample of the subject, or a measurement of a rate of change in CMV nucleic acid levels in the subject. In some embodiments, for any method described herein, the subject is pregnant, and the CMV therapy is administered to treat a fetus of the subject in utero. In some embodiments, for any method described herein, the subject is treated in the first half of pregnancy, within three weeks of infection, and/or prior to transmission of CMV to a fetus or appearance of CMV infection symptoms in the fetus. In some embodiments, for any method described herein, the CMV therapy is selected from the group consisting of an antiviral compound, a biologic, an immunotherapy, and termination of a pregnancy, or a combination of two or more of the listed items. In some embodiments, for any method described herein, the antiviral compound is selected from the group consisting of acyclovir, valacyclovir, valaciclovir, ganciclovir, valganciclovir, foscarnet, cytogam and cidofovir, or a combination of two or more of the listed items. In some embodiments, for any method described herein, the antiviral compound is selected from the group consisting of, valacyclovir, valaciclovir, ganciclovir, valganciclovir, foscarnet, and cidofovir. In some embodiments, for any method described herein, the CMV therapy is administered in a prophylactic dose that is lower than a lowest dose of the antiviral compound approved by a regulatory authority (e.g., the FDA or EMA) for primary infection. In some embodiments, for any method described herein, the CMV therapy is administered prior to transmission of CMV from the subject to a fetus of the subject. In some embodiments, the method further comprises monitoring subsequent samples of the subject after the start of the CMV therapy to detect changes in viral load or titer. A decrease in viral load or titer can indicate a response to the CMV therapy. The CMV therapy may be continued until there are two sequential negative high-sensitivity CMV tests. In some embodiments, for any method described herein, the immunoassay test also determines a presence or absence of anti-CMV IgM in the sample obtained from the subject. In some embodiments, for any method described herein, if the CMV nucleic acid sequences are detected, the method further comprises confirmatory testing. In some embodiments, for any method described herein, the confirmatory testing comprises a confirmatory immunoassay test for anti-CMV IgG and/or IgM, and/or a confirmatory high-sensitivity test for CMV nucleic acid in the subsequent sample or another sample. In some embodiments, for any method described herein, the confirmatory testing comprises detecting consecutive increases in quantitative CMV nucleic acid measurements or in CMV viral titer measurements. In some embodiments, for any method described herein, the confirmatory testing comprises a confirmatory immunoassay test to confirm a presence or absence of anti-CMV IgG in the subsequent sample or another sample. In some embodiments, for any method described herein, the confirmatory high-sensitivity test comprises detecting consecutive increases in quantitative CMV nucleic acid measurements and/or consecutive increases CMV viral titer measurements, indicating an active primary CMV infection. In some embodiments, for any method described herein, the method further comprises monitoring subsequent samples of the subject after a start of the CMV therapy to detect changes in viral load or titer. A decrease in viral load or titer after the start of the CMV therapy can indicate a response to the CMV therapy. The CMV therapy may be continued until there are two sequential negative high-sensitivity CMV tests. In some embodiments, for any method described herein, the sample comprises a blood sample, such as whole blood, serum, or plasma. In some embodiments, for any method described herein, the method further comprises the subsequent sample comprises stabilized preserved nucleic acids of the subject.

In some embodiments, a product combination for determining the presence of cytomegalovirus (CMV) in a subject is described. The product combination can comprise an immunoassay kit comprising a protein that binds specifically to anti-CMV IgG. The product combination can comprise a high sensitivity kit comprising nucleic acid that specifically hybridizes to a stabilized preserved CMV nucleic acid. In some embodiments, the immunoassay kit further comprises a protein that binds specifically to anti-CMV IgM. In some embodiments, the high-sensitivity kit comprises a receptacle for collection of stabilized preserved nucleic acid from the subject. In some embodiments, the high sensitivity kit comprises instructions for sending stabilized preserved nucleic acid obtained from the subject to a laboratory for testing. In some embodiments, the immunoassay kit is for use at a clinic. In some embodiments, the product combination further comprises a second immunoassay kit for determining a presence or absence of anti-CMV IgG and anti-CMV IgM. The second immunoassay kit can comprise a protein that binds specifically to anti-CMV IgG, and a protein that binds specifically to anti-CMV IgM. In some embodiments, the product combination further comprises instructions to use the second immunoassay kit up to three weeks after the high-sensitivity kit. In some embodiments, the product combination further comprises an avidity test kit. In some embodiments, the product combination further comprises instructions for using the avidity test kit up to three weeks after the second immunoassay kit. In some embodiments, the product combination further comprises a CMV therapy, the therapy selected from the group consisting of an antiviral compound, a biologic, an immunotherapy, and termination of a pregnancy, or a combination of two or more of the listed items. In the product combination of some embodiments, the CMV therapy comprises an antiviral compound selected from the group consisting of: acyclovir, valacyclovir, ganciclovir, valganciclovir, foscarnet, cytogam, and cidofovir, or a combination of two or more of the listed items. In the product combination of some embodiments, the CMV therapy comprises an antiviral compound selected from the group consisting of: valacyclovir, ganciclovir, valganciclovir, foscarnet, and cidofovir, or a combination of two or more of the listed items. In some embodiments, a kit comprises any of the product combinations described herein.

### BRIEF DESCRIPTION OF THE DRAWINGS

FIG. 1 is a flow diagram illustrating methods of detecting CMV infection (such as active primary CMV infection) in accordance with some embodiments herein.

FIG. 2 is a flow diagram illustrating methods of treating, inhibiting, or ameliorating CMV infection or a symptom thereof in accordance with some embodiments described herein.

FIG. 3 is a flow diagram illustrating methods of detecting CMV infection (such as active primary CMV infection) in accordance with some embodiments herein.

FIG. 4 is a flow diagram illustrating methods of detecting CMV infection (such as active primary CMV infection) in accordance with some embodiments described herein.

### DETAILED DESCRIPTION

Described herein are methods of detecting active primary CMV infection. The methods described herein can provide practical methods for identifying and triaging pregnancies at high risk for CMV-mediated fetal damage. The methods can be useful to detect CMV infection early, which can facilitate prompt clinical intervention, for example interventions to prevent or reduce the risk of prenatal CMV transmission to the fetus of an infected mother, and/or interventions to treat or ameliorate CMV infection or symptoms thereof. It is contemplated that the methods described herein can provide a rapid indication of whether the subject has a CMV infection, and thus can permit the administration of a CMV therapy prior to fetal-damaging CMV infection, but rather can inhibit or prevent fetal-damaging CMV infection. For example, monitoring at-risk subjects with high-sensitivity nucleic acid tests can detect the presence of CMV nucleic acid within hours of initiating the test, so that these subjects can be rapidly triaged for treatment to inhibit or prevent fetal-damaging CMV infection. Optionally, the methods can comprise screening a subject as having an occupational risk of CMV infection (it is contemplated that certain occupations, such as nurses, elementary or middle school teachers, nursing or psychiatric or home health aides, child care workers, teacher assistants, primary child care providers for preschoolers, and/or are exposed to one or more children under the age of five multiple times per week have a greater risk of CMV infection than the general population, for example parents or caregivers of multiple young children in daycare and/or preschool). The methods can comprise initial testing for anti-CMV IgG in a sample of a subject, such as a blood sample. If anti-CMV IgG is absent, the subject is determined to be at-risk for an active primary CMV infection. Subsequent samples of the at-risk subject (which can be collected from the subject at-home) can be monitored for CMV nucleic acids. If CMV nucleic acids are present, the subject is determined to have an active primary CMV infection. The subject can subsequently receive therapy for CMV infection or be recommended for therapy for CMV infection, for example receiving administration of an antiviral compound, a biologic, and/or an immunotherapy as described herein. It is contemplated that the combination of initial anti-CMV IgG testing and subsequent testing for CMV nucleic acids can provide information on the presence and status (e.g., primary versus secondary or recurrent) of the CMV infection, thus minimizing the risk of false positives from immunoassay testing alone, and providing more information than nucleic acid testing alone. Furthermore, initially screening for at-risk subjects using immunoassay tests can conserve health care resources, for example by avoiding costly nucleic acid testing when it is unlikely to be useful. Additionally, subsequent samples for nucleic acid testing can be collected in the absence of a health care provider, for example at the subject's home using a collection receptacle that stabilizes nucleic acids of the subject, thus avoiding the inconvenience and cost of a trip to the clinic and further increasing the likelihood that the subject will follow through with the subsequent round of testing. As such, it is contemplated that monitoring the subject for CMV nucleic acids can comprise monitoring samples that compromise stabilized, preserved nucleic acid.

In methods and uses of some embodiments, a CMV therapy (for example an antiviral compound such as acyclovir, valacyclovir, ganciclovir, valganciclovir, foscarnet, cytogam and/or cidofovir) can be for use in treating a subject determined to have an active, primary CMV infection as described herein. In methods and uses of some embodiments, the antiviral compound is selected from the group consisting of acyclovir, valacyclovir, ganciclovir, valganciclovir, foscarnet, cytogam, and/or cidofovir. In methods and uses of some embodiments, the antiviral compound is selected from the group consisting of valacyclovir, ganciclovir, valganciclovir, foscarnet and/or cidofovir. In methods and uses of some embodiments, in a subject determined to have an active, primary CMV infection as described herein, the CMV therapy can be administered to a fetus in utero. In methods and uses of some embodiments, in a subject determined to have an active, primary CMV infection as described herein, the CMV therapy can be administered prophylactically, for example to inhibit, reduce the likelihood of, or prevent transmission to a fetus. It is contemplated that the prophylactic administration of the CMV therapy can be at a lower dose than for conventional uses of that therapy, thus minimizing, reducing the likelihood of, or avoiding side effects, for example teratogenic effects associated with some compounds. In some embodiments, the CMV therapy comprises, consists essentially of, or consists of valacyclovir. It is noted that valacyclovir has a longer half-life than other antiviral compounds such as acyclovir, and thus can be amenable to lower dosage administration for inhibiting or preventing transmission of CMV to a fetus.

Conventional CMV testing detects either the presence of IgG (or IgM; IgG and IgM are typically expressed at different phases of the infection) or nucleic acid that is specific to CMV through a blood draw. It is contemplated that in methods, uses, and product combinations of some embodiments, IgG specific to CMV can be detected without necessarily detecting IgM. Testing for the presence of IgG (or IgM) is sensitive and economical, but provides limited information regarding the status and severity of the infection. Conventional nucleic acid testing is sensitive and can provide quantitative information about the status and severity of the infection, but cannot determine whether the infection is primary or secondary, and is substantially more expensive than testing for the presence of IgG (or IgM). Clinical intervention, whether pharmaceutical treatment, increased fetal monitoring, or termination, can depend on the rapid detection of an active CMV infection and the measurement of virus in the amniotic fluid. It is contemplated that conventional clinical management schedules and sample collection methodologies may limit the implementation of testing paradigms that have a reasonable chance of catching CMV infection when intervention can make a demonstrable impact on the subject. Methods, kits, and product combinations as described in some embodiments herein are contemplated to provide a demonstrable opportunity to catch an infection during a window when intervention can make a reasonable impact on the subject. In some embodiments, methods, kits and product combinations as described herein are useful for catching a primary CMV infection.

Detecting and diagnosing CMV infection in pregnant subjects is not conventionally practiced, has often been overlooked by clinicians in the US, and has yielded sub-standard results in the EU. Without being limited by theory, it is contemplated that the nuisance and cost of rounds of phlebotomy has contributed to infrequent use of conventional CMV tests. Methods, uses, and product combinations in accordance with some embodiments herein can provide timely and actionable information to clinicians, while minimizing the need for phlebotomy, or even trips by patients to the clinic. For example, performing high-sensitivity tests on subsequent samples comprising as described herein can yield rapid test results, which permit rapid intervention with a CMV therapy while there is a window of opportunity to inhibit or prevent fetal-damaging CMV infection. Furthermore, early and actionable information for clinicians can facilitate the use of therapies for CMV infection (such as antiviral compounds, biologics, and/or immunotherapies as described herein, for example valacyclovir), many are of which are in clinical testing and show promise as a means for intervening and reducing the impact of the disease on the baby.

### Occupational Risk of CMV Infection

It is noted that the methods, uses, and product combinations described herein can be used for any subject that may potentially have CMV infection, for example for any woman who is pregnant. However, it is contemplated that subjects with certain occupational exposures have a considerably higher risk of CMV infection than the general population. For example, pregnant women, health workers or child-care workers or women with multiple young children whose children are in daycare can have a high risk for CMV infection during early pregnancy. At least 15% of the population of pregnant women is at risk for exposure to CMV infection during early pregnancy. Accordingly, in accordance with methods, uses, and product combinations of some embodiments, a subject is screened as having an occupational risk of CMV infection. The “occupational risk of CMV infection” refers to a subject, who as a result of occupation, living arrangement, or routine activities is consistently exposed to young (middle school, elementary or preschool-age) children, and therefore is at an elevated risk for CMV infection compared to the general population. By way of example, if a subject is in a certain occupation, for example, a nurse, elementary or middle school teacher, nursing or psychiatric or home health aide, child care worker, teacher assistant, or primary child care provider for a preschooler, the subject can have an occupational risk of CMV infection. It is contemplated that in accordance with methods, uses, and product combinations of some embodiments, screening a subject as having an occupation risk of CMV infection can identify the subject as belonging to a high-risk subpopulation that is likely to benefit from the methods, uses, and product combinations as described herein. Thus, screening a subject as having an occupational risk of CMV infection can facilitate efficient use of testing resources (including laboratory time, clinician time, and test consumables), as well as increase the likelihood of compliance, since the subject having occupational risk will have a greater incentive to identify and detect CMV infection than a member of the general population.

### Anti-CMV IgG and Anti-CMV IgM

Immunoglobulin G (IgG) is the main type of immunoglobulin found in blood and extracellular fluid, and can respond to infection of body tissues. IgG molecules can be of any of several classes (e.g., IgG1, IgG2, IgG3) or subclasses. Without being limited by theory, IgG with specificity for an antigen is indicative of a long-pending, previous, or recurrent infection (for example a secondary infection).

Immunoglobulin M (IgM) is one of several types of immunoglobulin that are produced by vertebrates, and typically is associated with early infection or reactivation of a previously latent infection. As such, an IgM may have lower affinity for an antigen than an IgG for that same antigen, as the IgG may have undergone further affinity maturation.

IgG and IgM each have their customary and ordinary meaning as understood by one of skill in the art in view of this disclosure. They refer to full-length antibodies as well as functional fragments thereof, and can have antigen binding capability. The basic structural unit of a typical full-length IgG or IgM includes a tetramer and consists of two identical pairs of antibody chains, each pair having one light and one heavy chain. In each pair, the light and heavy chain variable regions are together responsible for binding to an antigen, and the constant regions are responsible for the antibody effector functions. Human IgG or IgM (respectively) can be detected in accordance with methods, kits, and product combinations of some embodiments herein using antibodies or binding fragments thereof, such as those specific for a human constant region of the IgG or IgM type (respectively).

As used herein, “anti-CMV IgG antibodies,” or more briefly “anti-CMV IgG” have their customary and ordinary meaning as understood by one of skill in the art in view of this disclosure. They refer to IgG that has a specificity for CMV. They can specifically or preferentially bind to CMV over other substances.

As used herein, “anti-CMV IgM antibodies,” or more briefly “anti-CMV IgM” have their customary and ordinary meaning as understood by one of skill in the art in view of this disclosure. They refer to IgM that has a specificity for CMV. They can specifically or preferentially bind to CMV over other substances.

### Samples

Samples from subjects can be used in accordance with methods, kits, and product combinations as described herein, for example for the detection of CMV infection. A “sample” has its customary and ordinary meaning as understood by one of skill in the art in view of this disclosure, and can refer to a sample obtained from an organism or from components (e.g., cells) of an organism, including cell cultures, or nucleic acid amplification of a sample directly or indirectly obtained from a subject. In some embodiments, the sample includes sections of tissues such as frozen sections. The sample can also be, for example, a physiological sample. In some embodiments, the sample is freshly collected. In some embodiments, the sample is preserved, for example via freezing, or via a preservative such as formalin. It will be understood that a sample in accordance with some embodiments can be apportioned, so that particular tests can be performed on portions of the sample (e.g., an entire sample does not need to be exhausted by one or more tests). It will further be understood that in accordance with some embodiments more than one sample can be collected from a single subject at or around the same time (for example, two blood draws from a particular stage), and can be pooled and/or tested separately, and that a “sample” also contemplates such samples collected at or around the same time from the subject.

Example kinds of samples that are suitable for methods, kits, and product combinations of embodiments herein can comprise, consist essentially of, or consist of whole blood, serum, plasma, blood cells (e.g., white blood cells), tissue or fine needle biopsy samples, sputum, cerebrospinal fluid, urine, peritoneal fluid, and pleural fluid, or cells therefrom, or a combination of two or more of these. In some embodiments, the sample comprises, consist essentially of, or consists of blood. In some embodiments, the sample comprises serum and/or plasma. As used herein, a “blood sample” (including variations of this root term) encompasses whole blood, as well as portions thereof, such as serum, plasma, white blood cells, red blood cells, cells of the hematopoietic lineage, or mixtures of two or more of the listed items. Thus, a blood sample of some embodiments comprises, consists essentially of, or consists of serum and/or plasma. Additionally, a blood sample of some embodiments comprises, consists essentially of, or consists of hematopoietic lineage cells. Depending on the type of test being performed (e.g., immunoassay test or high-sensitivity test), one of skill in the art can select a suitable sample type, for example a sample comprising immunoglobulin of the subject for use in immunoassay tests, or a sample comprising nucleic acids of the subject for use in a high sensitivity test. In some embodiments, the sample comprises, consist essentially of, or consists of serum, for example for the detection of anti-CMV IgG (and/or the detection of anti-CMV IgM). In some embodiments, the sample comprises, consists essentially of, or consists of plasma, for example for the detection of anti-CMV IgG (and/or the detection of anti-CMV IgM), and/or for the detection of CMV nucleic acids. In some embodiments, the sample comprises, consists essentially of, or consists of nucleated blood cells, saliva and/or buccal cells, for example for the detection of CMV nucleic acids. In some embodiments, the sample comprises, consist essentially of, or consists of serum, for example for the detection of anti-CMV IgG (and/or the detection of anti-CMV IgM).

In some embodiments, the subject is a human. In some embodiments, the subject is pregnant. For example, the method, kit, or product combination can be used to determine a presence of primary active CMV infection that may be transmitted to a fetus, and as such, the sample is collected from the pregnant subject. It can be advantageous, in methods, kits, and product combinations of some embodiments, to collect the sample as early in the pregnancy as possible. In some embodiments, the sample is collected during the first half of pregnancy (e.g., during the first trimester, and/or during the first half of the second trimester). In some embodiments, the subject is not pregnant. In some embodiments, the subject is at risk of having, is suspected of having, or is known to have a CMV infection.

In some embodiments, after a sample is collected from the subject at a first stage (for example a stage during a first half of pregnancy as described herein), a “subsequent sample” is obtained from the subject at a subsequent stage. In some embodiments, the subsequent stage is no more than 1, 2, 3, 4, 5, 6, 7, 8, 9, 10, 11, or 12 weeks after the first stage, including ranges between any two of the listed values, for example 1-12 weeks, 1-10 weeks, 1-8 weeks, 1-6 weeks, 1-4 weeks, 1-2 weeks, 2-12 weeks, 2-10 weeks, 2-8 weeks, 2-6 weeks, 2-4 weeks, 4-12 weeks, 4-10 weeks, 4-8 weeks, 4-6 weeks, 6-12 weeks, 6-10 weeks, or 6-8 weeks.

### Immunoassay Tests

Immunoassay tests can be useful for detecting anti-CMV IgG and/or anti-CMV IgM in methods, kits, and product combinations in accordance with some embodiments herein.

In some embodiments, an immunoassay test for detecting anti-CMV IgG comprises, consists essentially of, or consists of detecting the binding of one or more proteins that binds specifically to anti-CMV IgG, for example, CMV antigen and/or an antibody specific for IgG (such as human IgG). It is contemplated, that anti-CMV IgG can bind CMV, and can also be bound by an anti-IgG antibody (so as to detect the anti-CMV IgG). In some embodiments, the antibody is specific for anti-CMV IgG. In some embodiments, the proteins of the immunoassay test for detecting anti-CMV IgG comprise, consist essentially of, or consist of CMV antigen and an antibody specific for IgG, for example human IgG. The immunoassay test can further include reagents for detecting binding to anti-CMV IgG, for example secondary antibodies, detectable moieties (e.g., fluorophores, radiolabels, FRET pairs, enzyme-substrate pairs, enzymes, and the like as described herein), enzymes, or two or more of these.

In some embodiments, an immunoassay test for detecting anti-CMV IgM comprises, consists essentially of, or consists of one or more proteins that bind specifically to anti-CMV IgM, for example, CMV antigen and/or an antibody specific for IgM (such as human IgM). It is contemplated, for example, that anti-CMV IgM can bind CMV, and can also be bound by an anti-IgM antibody. In some embodiments, the antibody is specific for anti-CMV IgM. In some embodiments, the proteins of the immunoassay test for detecting anti-CMV IgM comprise, consist essentially of, or consist of CMV antigen and an antibody specific for IgM, for example human IgM. The immunoassay test can further comprise reagents for detecting binding to anti-CMV IgM, for example secondary antibodies, detectable moieties, enzymes, or two or more of these.

The immunoassay tests can further comprise suitable reagents for immunoassay testing, for example substrates (such as multi-well plates), buffers, secondary antibodies, detectable moieties, or two or more of these.

“Antibodies” have their customary and ordinary meaning as understood by one of skill in the art in view of this disclosure, and can refer to full-length antibodies as well as functional binding fragments. Examples of suitable antibodies formats for the identification of anti-CMV IgG, or for the identification of anti-CMV IgM include, but are not limited to, polyclonal antibodies, monoclonal antibodies, recombinantly produced antibodies, intrabodies, human antibodies, humanized antibodies, chimeric antibodies, synthetic antibodies, single-chain Fvs (scFv), Fab fragments, Fab fragments, disulfide-linked Fvs (sdFv) (including bi-specific sdFvs), and anti-idiotypic (anti-Id) antibodies, and epitope-binding fragments of any of the above. Multispecific antibodies can be specific for different epitopes of a polypeptide or may be specific for both a polypeptide as well as for a heterologous epitope, such as a heterologous polypeptide or solid support material. See, e.g., PCT Publication Nos. WO 93/17715; WO 92/08802; WO91/00360; WO 92/05793; Tutt, et al., J. Immunol. 147:60-69 (1991); U.S. Pat. Nos. 4,474,893; 4,714,681; 4,925,648; 5,573,920; 5,601,819; and Kostelny et al., J. Immunol. 148:1547-1553 (1992); each of which is incorporated herein by reference in its entirety. It is understood that an antibody or fragment thereof useful for detecting anti-CMV IgG in accordance with methods, kits, and product combinations of some embodiments herein will bind specifically to IgG (e.g., anti-CMV IgG) that is, it will bind with a greater affinity to the noted IgG than for other substances, such as anti-CMV IgM. For example, an antibody used in detecting anti-CMV IgG may be specific for human IgG, and have greater affinity for human IgG than for IgM. Antibodies specific for human IgG are readily available, for example polyclonal anti-IgG (such as Jackson ImmunoResearch Catalog No. 109-001-008O), rabbit monoclonal antibody EPR4421 to all four human IgG isotypes (Abcam Catalog No. ab109489), and mouse monoclonal antibody AbD27686 to human IgG1 (Bio-rad Catalog No. HCA285). It is understood that a protein (such as an antibody or fragment thereof) useful for detecting anti-CMV IgM in accordance with methods, kits, and product combinations in of some embodiments herein can bind specifically to anti-CMV IgM, that is, with a greater affinity to anti-CMV IgM than for other substances such as anti-CMV IgG. For example, an antibody used in detecting anti-CMV IgM may be specific for human IgM, and have greater affinity for human IgM than for IgG.

In some embodiments, the antibody specific for IgG, and/or the antibody specific for IgM is a non-human antibody, for example mouse, rat, guinea pig, rabbit, donkey, goat, horse, pig, and the like. In some embodiments, the antibody specific for IgG and/or the antibody specific for IgM is from a host of the same species as the subject. In some embodiments, the antibody specific for IgG and/or the antibody specific for IgM is from a host of a different species than the subject.

Immunoassay tests (also referred to herein as “immunological assays,” “immunoassays”), suitable for methods, kits, and product combinations of various embodiments herein can include radioimmunoassays and enzyme-linked immunoassays. A variety of immunoassay test formats, including competitive and non-competitive immunoassay formats, antigen capture assays and two antibody sandwich assays also are useful in accordance with the method, kits, and product combinations of embodiments herein (for a summary of various immunoassays, see Self and Cook, Curr. Opin. Biotechnol. 7:60-65 (1996), which is hereby incorporated by reference in its entirety). Exemplary immunoassays that are suitable in accordance with some embodiments herein include immunoassays, lateral flow assays, no-wash assays, sandwich immunoassays, competition immunoassays, ELISA, immunoblot assays, flow cytometry, immunohistochemistry, surface plasmon resonance, western blots, immunoblots, and the like.

In some embodiments of methods, kits, and product combinations as described herein, the immunoassay test comprises, consists essentially of, or consists of an enzyme-linked immunosorbent assay (ELISA). In some embodiments, ELISA comprises a sandwich ELISA. For example, a capture molecule of interest, such as a CMV antigen can be immobilized on a solid phase (such as a surface of a well in a multi-well plate) comprises by the assay environment. A sample that possibly comprises a CMV antigen can be contacted with the CMV antigen in the assay environment, so that anti-CMV IgG and/or IgM present in the sample can bind the CMV antigen. To detect the present of anti-CMV IgG, the assay environment can be contacted with an anti-human IgG antibody. The anti-human IgG antibody can either be detected directly (for example, by labeling the anti-human IgG antibody with a detectable moiety), or indirectly, for example by using a secondary antibody labeled with a detectable moiety. To detect the presence of anti-CMV IgM, the assay environment can be contacted with an anti-human IgM antibody. The anti-human IgM antibody can either be detected directly (for example, by labeling the anti-human IgM antibody with a detectable moiety), or indirectly, for example by using a secondary antibody. Examples of suitable detectable moieties include, but are not limited to, fluorophores, quantum dots, enzymes, substrates, metal particles (such as nanoparticles), radiolabels, dyes, and the like.

In some embodiments of methods, kits, and product combinations as described herein, the immunoassay test comprises, consists essentially of, or consists of a competition immunoassay (sometimes also referred to a “competitive immunoassays” and the like). By way of example, competition immunoassays can be used for ELISA, lateral flow systems, no-wash assays, and the like. In some embodiments, the ELISA comprises competitive ELISA (which may also be referred to as competition ELISA).

In some embodiments of methods, kits, and product combinations as described herein, the immunoassay test comprises, consists essentially of, or consists of a no-wash assay. A no-wash assay can detect the presence or absence of a molecule of interest through the detection of a signal (or the absence of a signal) indicating the association of two different detectable moieties. In some embodiments, the two different detectable moieties are a FRET pair. In some embodiments, the FRET pair comprises a donor moiety and an acceptor moiety. In some embodiments, the two different detectable moieties are a fluorophore quencher pair. The no-wash system can include a first antigen (for example CMV) and a second antigen binding molecule, each of which bind to the same target at a different site, for example anti-human IgG antibody or anti-human IgM antibody. As such, in some embodiments, the first antigen binding molecule and the second antigen binding molecule can be bound to the same target at the same time. The signal (or absence of signal) produced by the association of the detectable moiety of the first antigen binding molecule and that of the second antigen binding molecule can indicate that both antigen binding molecules have bound to the target.

In some embodiments, a no-wash assay includes a plurality of different detection assays in a single reaction environment. In some embodiments a first binding agent—detectable moiety pair each comprising a different member of a first FRET pair, and a second binding agent—detectable moiety pair each comprising a different member of a second FRET pair that is different from the first FRET pair are assessed in the same reaction environment. In some embodiments, at least two different FRET pairs, for example 2, 3, 4, 5, 6, 7, 8, 9, or 10, FRET pairs are assessed in the same reaction environment. As such, a multiplex no-wash assay can be performed.

In some embodiments, the reaction environment of a no-wash assay comprises a well in a multi-well format plate, a test tube, a cuvette, a flask, or the like. In some embodiments, the reaction environment of a no-wash system is configured for detection by an electromagnetic radiation detector. As such, in some embodiments, at least one surface of the reaction environment is penetrable to electromagnetic radiation. In some embodiments, the electromagnetic radiation has a wavelength in the visible spectrum. In some embodiments, the electromagnetic radiation has a wavelength in a fluorescent excitation and emission spectrum.

An immunoassay test that determines the subject molecule (e.g., anti-CMV IgG or anti-CMV IgM) to be present in the sample may also be referred to herein as a test result that is “positive” for that molecule (e.g., “positive for anti-CMV IgG” or “positive for anti-CMV IgM”). By way of example, in some embodiments, anti-CMV IgG is determined to present in a sample if the immunoassay test indicates a greater quantity of signal for anti-CMV IgG than in a control sample known to be absent for anti-CMV IgG. By way of example, in some embodiments, anti-CMV IgG is determined to present in a sample if the immunoassay test indicates a greater quantity of signal than the limit of detection for the immunoassay test. If an immunoassay test indicates a presence of anti-CMV IgG in accordance with some embodiments herein (or “positive for anti-CMV IgG”), the subject can be determined to have a low likelihood of being at-risk for an active primary IgG infection.

An immunoassay test that determines the absence of the subject molecule (e.g., anti-CMV IgG or anti-CMV IgM) from the sample may also be referred to herein as a test result that is “negative” for that molecule (e.g., “negative for anti-CMV IgG” or “negative for anti-CMV IgM”). By way of example, in some embodiments, anti-CMV IgG is determined to absent from a sample if the immunoassay test indicates a comparable or lower quantity of signal for anti-CMV IgG than in a control sample known to be absent for anti-CMV IgG. By way of example, in some embodiments, anti-CMV IgG is determined to absent from a sample if the immunoassay test indicates a lower quantity of signal than the limit of detection for the assay. If an immunoassay test indicates an absence of anti-CMV IgG in accordance with some embodiments herein (or “negative for anti-CMV IgG”), the subject can be determined to be at-risk for an active primary IgG infection.

In some embodiments, the immunoassay test balances sensitivity and selectivity so as to balance the risk of the immunoassay test yielding false negatives and false positives. It is appreciated herein that if sensitivity is increased (so as to reduce the risk of a false negative), selectivity can decrease. It is appreciated herein that if selectivity is increased (so as to reduce the risk of a false positive), sensitivity can decrease. Accordingly, in some embodiments herein, the detection threshold of the immunoassay is calibrated so that a limit of detection, or detected level of binding to anti-CMV IgG or anti-CMV IgM balances sensitivity and selectivity, and minimizes the risk of a false negative and/or a false positive. For example, different amounts of binding reagent can be calibrated on a standard binding curve, and be compared to standard binding curves of control samples known to be positive for active primary CMV infection (these can include electronically or optically stored controls, as well as physical samples), and/or control samples known to be negative for active primary CMV infection (these can include electronically or optically stored controls, as well as physical samples).

### High-Sensitivity Testing

“High-sensitivity testing” can be performed on samples in accordance with methods, product combinations, and kits of some embodiments herein. As used herein, “high-sensitivity” testing refers to detection of CMV nucleic acids, such as DNA or RNA. It is noted that CMV is generally a DNA virus that can propagate through DNA replication, and that RNAs encoded by CMV DNA can also be expressed by infected cells. As used herein “CMV nucleic acid” or “CMV nucleic acid sequences” can refer to nucleic acids of CMV virus, as well as transcripts, copies, and amplicons thereof. Example CMV nucleic acid sequences include, but are not limited to sequences of HCMV UL83, sequences of HCMV glycoprotein B, sequences of CMV pp67, 5′-GGACGTATCCACCTCAGGTACACA-3′ (SEQ ID NO: 1); 5′-CGTGTTTCACAAACTGCACCAGTACCA-3′ (SEQ ID NO: 2); 5′-AAGTACCCCTATCGCGTGTG-3′ (SEQ ID NO: 3); 5′-CGCTCTTTTTGTCAGTCTGAATCGACCC-3′ (SEQ ID NO: 5); and/or 5′-AAAAGAGACCGCGTCTCTGG-3′ (SEQ ID NO: 4), including combinations of two or more of the listed sequences. It is noted that primers of SEQ ID NOs: 1 and 2 hybridize to a nucleic acid of the CMV gene encoding the membrane glycoprotein US9 (SEQ ID NO: 9)(See SEQ ID NO: 6 for the encoded glycoprotein US9). A primer of SEQ ID NO: 3 hybridizes to the CMV gene encoding the UL55 envelope glycoprotein B (SEQ ID NO: 10)(See SEQ ID NO: 7 for the encoded glycoprotein B). Primers of SEQ ID NOs: 4 and 5 hybridize to the CMV gene encoding the non-coding RNA, RNA4.9 (SEQ ID NO: 8). Sequences of the CMV genome are readily available, for example as sequence of human betaherpesvirus 5 (a.k.a. human cytomegalovirus, or HCMV) strain HER1 provided under GenBank Accession No. MF084224, or the CMV genome sequence of human herpesvirus 5 strain AD169 provided under GenBank Accession No. FJ527563. In view of these sequences, additional primers for amplifying sequences of the HCMV genome can readily be prepared. Suitable primers for amplifying any of the CMV sequences described herein can be designed, accounting for factors such as stringency of hybridization and melting temperature (Tm). Extensive guidance on nucleic acid hybridization and amplification protocols can be found, for example, in of nucleic acids is found in Tijssen (1993) Laboratory Techniques in Biochemistry and Molecular Biology—Hybridization with Nucleic Acid Probes, Part I, Chapter 2 (Elsevier, New York); Ausubel et al, eds. (1995) Current Protocols in Molecular Biology, Chapter 2 (Greene Publishing and Wiley-Interscience, New York); and Sambrook et al. (2012) Molecular Cloning: A Laboratory Manual (4th ed., Cold Spring Harbor Laboratory Press, Plainview, N.Y.). For example, Mendez, J. C., et al. J Clin Microbiol. 36: 526-30 (1998), hereby incorporated by reference in its entirety, designed and confirmed primer pairs for amplification and early detection of human CMV genome using the immediate-early antigen 1 (IEA1) gene (Primer P1=SEQ ID NO: 11; Primer P2=SEQ ID NO: 12) and major immediate-early (MIE) gene (Primer P1=SEQ ID NO: 13; Primer P2=SEQ ID NO: 14) of CMV AD169. By way of example, a primer pair for amplifying CMV sequences can comprise SEQ ID NO: 3 as a first primer and SEQ ID NO: 4 as a second primer. In some embodiments, the primers are designed such that the Tm of one primer in the set is within 2° C. of the Tm of the other primer in the set. In some embodiments, two or more of the listed sequences are used as primers and/or probes for high-sensitivity testing, for example quantitative PCR. The high-sensitivity testing can have a low limit of detection, and in some embodiments, can detect as little as a single CMV nucleic acid in a sample. In some embodiments, the high sensitivity testing comprises nucleic acid amplification, for example, polymerase chain reaction, rolling circle amplification, for example quantitative PCR such as real-time PCR or reverse transcription polymerase chain reaction (RT-PCR), or isothermal amplification (such as multiple displacement amplification (MDA)). Particular CMV nucleic acids can be amplified, and/or nucleic acids in general can be amplified non-specifically. In some embodiments, the high-sensitivity testing comprises microarray analysis for CMV nucleic acids. In some embodiments, the high-sensitivity testing comprises nucleic acid sequencing. Sequences of nucleic acids as described herein can be identified. In some embodiments, the high-sensitivity testing is quantitative, and, for example, can be used to determine levels of CMV nucleic acids, and/or changes in levels of CMV nucleic acids over time (e.g. for subsequent samples collected from a subject according to intervals as described herein).

The high sensitivity testing can be performed on sample of the subject comprising stabilized preserved nucleic acid from the subject. Without being limited by theory, it is contemplated that DNA is stable in most circumstances, but that in some embodiments, an additive such as a buffer may be combined with the sample at the time of collection, for example if the sample is destined for molecular or nucleic acid analysis. It is contemplated that such stabilized, preserved nucleic acid can be collected from subject in home collection tests, and thus can be obtained without the need for a phlebotomist or health care practitioner (such as a nurse or physician) to be present. In some embodiments, an additive, chelator, or an anticoagulant can further be present with the sample. In some embodiments, the sample is a blood sample. In some embodiments, a buffer such as ethylenediaminetetraacetic acid (EDTA) is present with the sample, for example, K2 EDTA. The buffer such as EDTA may stabilize the sample. In some embodiments, EDTA and salts are present in a sample. In some embodiments a sample collection receptacle comprises EDTA and/or salts. Without being limited by theory, it is contemplated that the option of high-sensitivity testing on samples obtained from the comfort of subjects' homes can increase compliance by subjects. Sample collection receptacles of some embodiments comprise stabilizers such as buffers, which can help maintain the sample until it is tested at a testing facility. Examples of suitable collection receptacles include OnDemand™ receptacles (commercially available from Tasso, Inc.) and TAP receptacles (commercially available from Seventh Sense Biosystems). In the high-sensitivity testing of methods, kits, and product combinations of some embodiments, a sample of the subject comprising nucleic acids (for example, blood, saliva, buccal cells, or the like) can be collected in a collection receptacle. The collection receptacle can comprise reagents for stabilizing the nucleic acids, for example buffers and/or buffer salts. In some embodiments, cells remain un-lysed in the collection receptacle for a period of time, even if no other agents are added. For example, the cells can remain un-lysed for a period of at least one week, two weeks, or three weeks. The collection receptacle comprising the sample comprising stabilized nucleic acids can then be provided for detection of CMV nucleic acids, for example, nucleic acid amplification (e.g., polymerase chain reaction, or rolling circle amplification, quantitative PCR such as real-time PCR, isothermal amplification (such as MDA), or reverse transcription polymerase chain reaction), microarray analysis, or nucleic acid sequencing.

In accordance with methods, kits, and product combinations of some embodiments herein, high sensitivity testing is performed on subsequent samples of subjects that are determined to be at-risk for a presence of active primary CMV infection. Without being limited by theory, it is contemplated that if a subject is not at-risk for a primary CMV infection (for example, if they are positive for anti-CMV IgG, such as high-avidity anti-CMV IgG), there is little need to incur the cost and effort of high-sensitivity testing. As such, health care resources can be conserved, and inconvenience to the subject can be minimized. It is noted, however, that in some instances, certain categories of IgG-positive women may be at-risk for active primary CMV infection, for example if these women are immunocompromised, for example as a result of HIV infection, or treatment with immunosuppressants. Accordingly, in some embodiments, a woman who is immunocompromised is considered to be at-risk for a primary CMV infection (regardless of serotype), and accordingly, is considered to be “at risk” in accordance with embodiments described herein. Thus, a subject who is immunocompromised may undergo high sensitivity testing is performed on subsequent samples of the subject as described herein, while testing for anti-CMV immunoglobulin (such as IgG) may optionally be omitted for this class of subject. In some embodiments, a health care professional orders a series of two or more tests in a single order (for example, a first stage test, and/or one or more subsequent-stage tests). Accordingly, samples can be collected—from the subject in a non-clinical setting (the comfort of the subject's own home), and without repeated visits with the health care professional. For convenience, sample collection in a non-clinical setting may be referred to herein as “at-home.” However, it will be appreciated that “at-home” sample collection is not limited to collection that is literally performed in a home, but rather can be performed in any suitable location, with the proviso that the presence of a licensed health care professional, such as a phlebotomist, nurse, or a physician is not required for the collection. By way of example, in some embodiments a health care professional orders all the required serial tests in a single setting. In some embodiments, the health care professional orders some or all of the tests on an as-needed basis.

The high-sensitivity testing of methods, kits, and product combinations of some embodiments herein can be performed on subsequent samples of at-risk subjects. The subsequent samples can be obtained at a subsequent stage (subsequent to the first stage). In some embodiments, a first high sensitivity test is performed on a sample collected from the subject about 1, 2, 3, 4, 5, 6, 7, 8, 9 or 10 weeks after the first-stage sample was collected from the subject, including ranges between any two of the listed values, for example 1-10, 1-7, 1-6, 1-5, 1-4, 1-3, 1-2, 2-10, 2-7, 2-6, 2-5, 2-4, 2-3, 3-10, 3-7, 3-6, 3-5, 3-4, 4-10, 4-7, 4-6, 4-5, 5-10, 5-7, or 5-6 weeks after. In some embodiments, a first high sensitivity test is performed on a subsequent sample that was collected from the subject about 3 weeks after the first-stage sample was collected from the subject. In some embodiments, two or more subsequent samples are collected from the subject 1, 2, 3, 4, 5, 6, 7, 8, 9, or 10 weeks apart from each other such that high sensitivity tests are done on samples collected 1, 2, 3, 4, 5, 6, 7, 8, 9, or 10 weeks apart from each other, including ranges between any two of the listed values, for example 1-10, 1-7, 1-6, 1-5, 1-4, 1-3, 1-2, 2-10, 2-7, 2-6, 2-5, 2-4, 2-3, 3-10, 3-7, 3-6, 3-5, 3-4, 4-10, 4-7, 4-6, 4-5, 5-10, 5-7, or 5-6 weeks apart. Accordingly, it is contemplated that in accordance with methods, kits, and uses of some embodiments, at least 1, 2, 3, 4, 5, 6, 7, 8, 9, 10, 11, 12, 13, 14, or 15 subsequent samples (including ranges between any two of the listed values, for example 1-15, 1-10, 2-15, 2-10, 5-15, or 5-10) can be collected. The collected subsequent samples can then be tested with high-sensitivity tests as described herein. In some embodiments, subsequent samples are collected two to three weeks apart from each other. The subsequent samples of some embodiments can be tested at the same time (for example, if the rate of change in levels of CMV is being measured), or in real-time (as they become available). In some embodiments, subsequent samples are collected from the subject during the second half of pregnancy (e.g., during the second half of the second trimester, and/or during the third trimester). Without being limited by theory, while risk of a major disability in the child from CMV infection may be lower during the second half of pregnancy, there may still be health benefits to treatment during this stage.

In some embodiments, a subsequent sample comprising nucleic acids is collected from a subject at a location that is clinical or non-clinical. In some embodiments, a subsequent sample comprising nucleic acids of the subject is collected in a location that is non-clinical (e.g., the subject's home). The sample can be transmitted (e.g., via mail or courier) to a testing facility. In some embodiments, the subsequent sample comprising nucleic acid sequences from a subject is collected without the presence of a licensed health care professional, such as a phlebotomist, nurse, or a physician. In some embodiments, such a sample is collected in a sample collection receptacle that comprises reagents for the stabilization and preservation of RNA and/or DNA (including those of CMV), for example EDTA. In some embodiments, such a sample is sent directly to a lab for high-sensitivity testing for the presence of nucleic acid sequences specific to CMV. In some embodiments, the subsequent sample is collected in a clinical setting.

High-sensitivity testing in accordance with methods, kits, and product combinations of embodiments herein can indicate a presence of CMV if the sample comprises CMV nucleic acids. A high sensitivity test result indicating presence of CMV nucleic acids may be referred to as “positive” for CMV nucleic acids. For example, the high-sensitivity test can be “positive” when signal for a CMV nucleic acid is detected at a level greater than the limit of detection, and/or when signal for a CMV nucleic acid in a sample is greater than for a control sample known to be negative for CMV nucleic acids. In some embodiments, a sample is determined to comprise CMV nucleic acids when CMV nucleic acids are present above the limit of detection in a high-sensitivity test. In some embodiments, a sample is determined to comprise CMV nucleic acids when a high-sensitivity test indicates a greater quantity of CMV nucleic acids in that sample compare to a control sample known to be negative for CMV nucleic acids.

In some embodiments, subsequent testing can comprise detecting consecutive increases in quantitative nucleic acid sequences measurements specific to CMV. If consecutive increases are detected over a specified period, the high-sensitivity testing can indicate a presence of CMV. In some embodiments, consecutive increases in CMV viral titer measurements indicate an active, primary infection. In some embodiments, subsequent high-sensitivity testing can be performed after the subject has begun to receive a CMV therapy as described herein, for example oral administration of an antiviral such as valacyclovir. A decrease in CMV viral load or viral titer can indicate a response to the CMV therapy. The subsequent high-sensitivity test can monitor the subject's response to the CMV therapy. By way of example, the CMV therapy may be continued until there are two sequential negative high-sensitivity CMV tests.

Confirmatory and/or orthogonal as described herein can also comprise, consist essentially of, or consist of high sensitivity testing.

### Confirmatory Testing

In accordance with methods, kits, and product combinations of some embodiments, the positive detection of CMV nucleic acid in a high-risk subject sample can be followed by subsequent confirmatory nucleic acid testing and/or immunoassay testing. Optionally, the confirmatory testing can be orthogonal to the testing approaches (e.g. immunoassay tests and/or high sensitivity tests) that have already been performed. It is contemplated, however, that in accordance with methods, uses, and product combinations of some embodiments, early detection of active primary CMV infection is desirable so that a CMV therapy (as described herein) can be timely administered to inhibit, reduce the likelihood, or prevent transmission of CMV to a fetus, or to treat or inhibit CMV infection in the fetus in utero. As such, in some embodiments, confirmatory testing is performed within 3 weeks, 2 weeks, 1 week, 6, 5, 4, 3, 2, or 1 days of the positive detection of CMV nucleic acid in an at-risk subject sample. By way of example, further (confirmatory) immunoassay tests for anti-CMV IgG and/or IgM can be performed, and/or further high-sensitivity tests for CMV nucleic acid (which may test for CMV nucleic acid sequences that are the same or different as those that were already tested) can be performed. Advantageously, a confirmatory test that delivers a rapid result can facilitate rapid referral of the subject for CMV therapy. It is contemplated that high-sensitivity tests as described herein, for example, qualitative or quantitative PCR can yield results in a matter of hours from the start of the test, and thus can facilitate rapid referral for CMV therapy as described herein. Accordingly, in some embodiments, the confirmatory test comprises nucleic acid amplification, such as quantitative of qualitative PCR, rolling circle, or isothermal amplification. In some embodiments, the confirmatory test comprises an ELISA. If confirmatory testing is positive, the subject can be determined to have an active primary CMV infection. Optionally, the subject can be referred to an invasive amniocentesis procedure to determine the magnitude of infection. Clinical studies have shown that high viral titers in amniotic fluid result in increased risk of fetal disease and complications. Therefore, administered CMV therapy can be tailored to the relative risk associated to the fetus as determined by viral titers.

### Avidity Tests

In accordance with methods, product combinations, and kits of some embodiments, an avidity test is performed, for example when a presence of IgG and a presence of IgM is detected. The avidity testing can help to indicate whether the subject is at-risk for active primary CMV infection.

“Avidity” has its customary and ordinary meaning as understood by one of skill in the art in view of this disclosure. It refers to strength with which an antigen binding molecule (such as IgG or IgM) binds to antigenic epitopes expressed by a given protein. Without being limited by theory, IgG matures gradually during the months following primary infection. Low CMV IgG avidity is an accurate indicator of primary infection within the preceding three to four months, whereas high avidity excludes primary infection within the preceding three months.

Avidity testing can may provide useful information regarding timing of infection. IgG produced following primary CMV infection can have low avidity (low binding strength). About two to four months following infection, IgG can mature to high-avidity (high binding strength). Avidity tests in accordance with methods, kits, and product combinations of embodiments herein can be used to ascertain whether the IgG is low avidity (indicting recent infection) or high avidity (past infection). In some embodiments, the avidity test differentiates between active primary and past infections.

In some embodiments, avidity is measured by ELISA. In some embodiments, measuring IgG avidity comprises an ELISA comparing binding of the subject's IgG to CMV in in the presence versus absence of a chaotropic agent, typically a mild chaotropic agent, such as urea, DEA, thyiocyanate, or the like. In some embodiments, an amount of bound antibody (e.g., via a dilution curve) in the ELISA in the presence of a chaotropic ion can be compared to an amount of bound antibody (e.g., via a dilution curve) in the ELISA in the absence of the chaotropic ion. ELISA-based CMV IgG avidity kits are commercially available, such as the ARUP laboratories “Cytomegalovirus, IgG Avidity” kit. In some embodiments, IgG avidity is measured by an affinity constant of the IgG to CMV, for example a K_{D}. The affinity constant can be calculated using methods such as surface plasmon resonance, which can be performed, for example, using a BIACORE device. The avidity of the IgG of the subject can be compared to a control (for example, a sample of a subject known to comprise high avidity anti-CMV IgG), and/or can be compared to a threshold value such as an affinity constant. In some embodiments, the IgG of the subject is considered high avidity when it binds more tightly than a predetermined affinity constant.

In some embodiments, an avidity test as described herein is performed on a control sample. In some embodiments, the control sample is of a subject who is known to have an active, primary CMV infection. In some embodiments, the control sample is of a subject who is known to have a developed CMV infection. A “developed CMV infection” refers to a CMV infection that produces high-avidity CMV, and typically represents an infection that initiated at least about two to four months ago, for example at least about three months ago. A “CMV-developed control” refers to a control that has a developed CMV infection. In some embodiments, the control sample is taken from a subject who does not have a CMV infection (e.g., a CMV-naïve control), which can represent a negative control.

In methods, kits, and product combinations of some embodiments, if the sample of the subject is positive for anti-CMV IgG and positive for anti-CMV IgM, the subject's IgG having a lower avidity to CMV than that of a CMV-developed control indicates that the subject is at-risk for an active primary CMV infection. High-sensitivity testing can then be performed as described herein.

In methods, uses, kits, and product combinations of some embodiments, if the sample of the subject is positive for anti-CMV IgG and positive for anti-CMV IgM, the subject IgG having a comparable or higher avidity to CMV than that of a CMV-developed control indicates that the subject is not at-risk for an active primary CMV infection. Such a subject may have a developed CMV infection, or may have had a previous CMV infection.

### Methods of Determining a Presence of Active Primary Cytomegalovirus (CMV) Infection

In some embodiments, a method of determining a presence of active primary CMV infection is described. The method can comprise receiving a sample (for example, a blood sample) obtained from a subject at a first stage. Optionally, the method comprises screening the subject as having an occupational risk of CMV infection prior to receiving the sample. The method can further comprise determining a presence or absence of anti-CMV IgG in the sample. The presence or absence of anti-CMV IgG can be determined using an immunoassay test as described herein. An absence of anti-CMV IgG indicates that the subject is at-risk for a presence of CMV infection. If the subject is at-risk, the method can further comprise monitoring a subsequent sample of the subject obtained from the subject at a subsequent stage. The subsequent sample can comprise nucleic acid from the subject, which can optionally be stabilized and preserved. The monitoring can comprise detecting a presence or absence of CMV nucleic acid sequences in the subsequent sample using a high sensitivity test as described herein. An absence of anti-CMV IgG in the sample and a presence of CMV nucleic acid in the subsequent sample (and, optionally a presence of anti-CMV IgM) indicate a presence of CMV, such as an active primary CMV infection. The methods described herein can be advantageous because a subject is more likely to follow through to determine with the testing regimen to determine a likelihood of CMV infection if at least some portions of the testing can be done without clinical visits, but rather in the comfort of the subject's own home. A presence of anti-CMV IgG indicates that the subject is at a low likelihood of being at-risk for a presence of CMV, though optionally, further testing for the presence of absence of anti-CMV IgM and/or avidity testing can be performed as described herein.

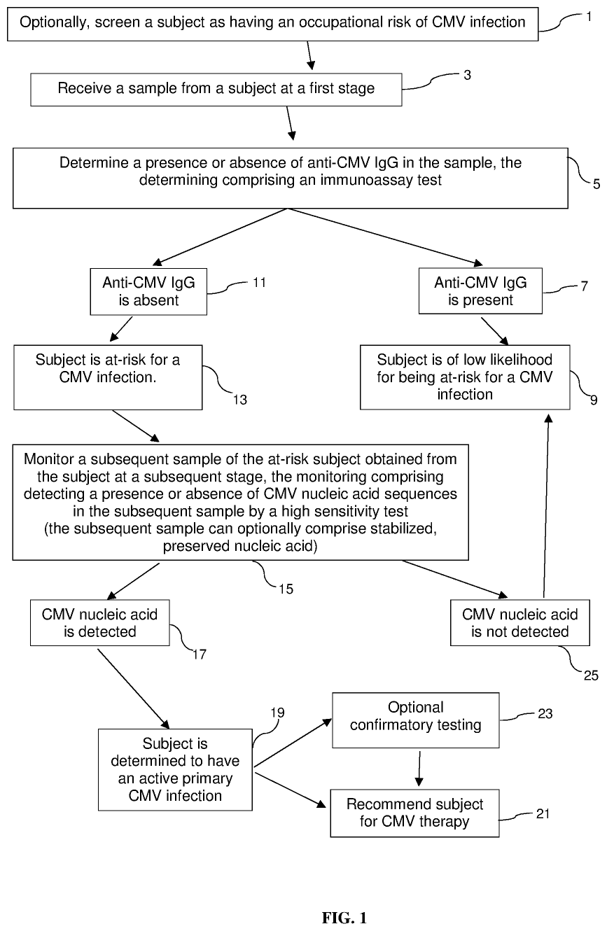

FIG. 1 is a flow diagram illustrating a method of determining a presence of active primary CMV infection according to some embodiments herein. Optionally, the method can include screening the subject as having an occupational risk of CMV infection 1. Examples of occupational risks of CMV infection include a nurse, elementary or middle school teacher, nursing or psychiatric or home health aide, child care worker, teacher assistant, primary child care provider for a preschooler (such as a parent or other caregiver who has one or more children under the age of five who are in preschool or day care), and/or a woman exposed to one or more children under the age of five multiple times per week. The method can include receiving a sample from a subject at a first stage 3. By way of example, the first sample can comprise serum, plasma, or (whole) blood. The method can comprise determining a presence or absence of anti-CMV IgG in the sample, the determining comprising an immunoassay test 5. An absence of anti-CMV IgG can indicate that the subject is at-risk for a CMV infection (for example, an active primary CMV infection) 9, and thus, of higher risk for developing or having a fetal-damaging CMV infection. If anti-CMV IgG is present 7, the subject can be of low likelihood for being at-risk for a CMV infection (for example, an active primary CMV infection) 9, and thus, of lower risk for developing or having a fetal-damaging CMV infection. If anti-CMV IgG is absent 11, the subject can be at-risk for an active primary CMV infection 13. Accordingly, the method can comprise monitoring a subsequent sample of the at-risk subject obtained from the subject at a subsequent stage, the monitoring comprising detecting a presence or absence of CMV nucleic acid sequences in the subsequent sample by a high sensitivity test 15. The subsequent sample can optionally comprise stabilized, preserved nucleic acid. The subsequent stage can be at least 1 week after the first stage, for example at least 1, 2, 3, 4 or 5 weeks after the first stage, including ranges between any two of the listed values as described herein. If CMV nucleic acid is detected by the high-sensitivity test 17, the subject can be determined to have an active, primary CMV infection 19. Accordingly, the subject can be recommended for CMV therapy 21, for example an antiviral compound such as valacyclovir as described herein. Optionally, confirmatory testing can be performed 23 prior to recommending the subject for CMV therapy. It is contemplated that the CMV therapy can be useful to inhibit, prevent, or reduce the likelihood of CMV transmission to a fetus (for example, in the case of a pregnant subject), and/or to treat or inhibit CMV infection in utero. Accordingly, confirmatory testing can be completed within 3, 2, or 1 weeks, so as to facilitate intervention with CMV therapy prior to transmission to the fetus. If CMV nucleic acid is not detected 25, the subject can be of low likelihood for being at-risk for active primary CMV infection 9. In some embodiments, the method comprises collecting the sample (e.g., blood sample). It is further contemplated that in some embodiments, for any method described in FIG. 1, the sample can be collected, and the determination of the presence of anti-CMV IgG 5 and/or monitoring the subsequent sample 15 can be instructed to a third party (rather than directly performed by the entity that collects the samples), and/or the results of the determining and/or monitoring can be received from a third party. Accordingly, wherever a method of determining a presence of active primary CMV infection is described herein, adjustments comprising collecting the sample, instructing the determining and/or monitoring, and/or receiving the results of the determining and/or monitoring are expressly contemplated as well.

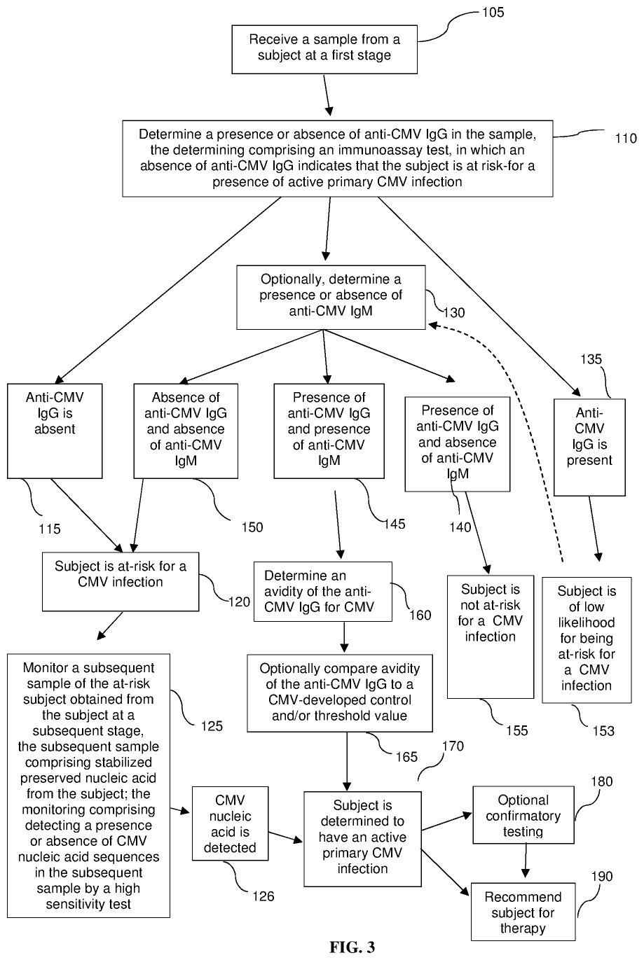

FIG. 3 is a flow diagram illustrating a method of determining a presence of active primary CMV infection according to some embodiments herein. In some embodiments, a sample of a subject (e.g., a blood sample) is received 105. In some embodiments, a presence or absence of anti-CMV IgG in the sample is determined 110. The presence or absence of the anti-CMV IgG can be determined using an immunoassay test as described herein, in which an absence of anti-CMV IgG indicates that the subject is at-risk for a CMV infection (for example, an active primary CMV infection) 9, and thus, of higher risk for having or developing a fetal-damaging CMV infection. When there is an absence of IgG 115, the subject whose sample is received can be determined to be at-risk for a CMV infection (for example, an active primary CMV infection) 120, and thus, at-risk for having or developing or having a fetal-damaging CMV infection. If the subject is at-risk, a subsequent sample of the at-risk subject can be monitored 125. The subsequent sample can comprise stabilized preserved nucleic acid from the subject, for example in a blood or buccal sample. The monitoring can comprise detecting a presence or absence of CMV nucleic acid sequences in the subsequent sample by a high sensitivity test. If the CMV nucleic acid sequences are detected by the high sensitivity test 126, the subject can be determined to have an active primary CMV infection 170. If the subject is determined to have an active primary CMV infection, optionally, confirmatory testing can be performed. 180. Optionally, the subject can be recommended for a therapy for of CMV infection 190. Optionally, the subject (or the subject's fetus, if applicable) can then be treated for CMV infection as described herein. In some embodiments, the presence or absence of anti-CMV IgG is determined 110, and anti-CMV IgG is present in the sample of the subject 135. When the anti-CMV IgG is present 135, the subject is determined to have a low likelihood of being at risk for a CMV infection (for example, an active primary CMV infection) 153, and thus, of lower risk for having or developing a fetal-damaging CMV infection (being determined to have a “low likelihood” of being at-risk for active primary CMV infection as used herein is a risk category in which active primary CMV has not been ruled-out, but based on information obtained thus far, for example a presence of anti-CMV IgG, the subject has a lower likelihood of having an primary CMV infection than a comparable untested subject). Optionally, for the low-likelihood subject, an avidity of sample anti-CMV IgG for CMV can be determined. It is noted that if the anti-CMV IgG is present, and the avidity of the anti-CMV IgG for CMV is low, there is a possibility of an early active infection, subsequent samples of such a subject may be monitored 125. Optionally, for the low-likelihood subject of 153, a presence or absence of anti-CMV IgM can be determined 130 to provide further information on whether the subject is at-risk for active primary CMV infection. In some embodiments, in addition to determining presence or absence of anti-CMV IgG 110, optionally, a presence or absence of anti-CMV IgM 130 can be determined as well. When there is an absence of IgG and an absence of IgM 150, the subject can be determined to be at at-risk for active primary CMV infection 120. If the subject is at-risk, a subsequent sample of the at-risk subject (obtained at a subsequent stage that can be 1, 2, 3, 4, or 5, weeks after the initial phase, including ranges between any two of the listed values, for example, 1-3, 1-5, 2-3, 2-5, or 3-5 weeks) can be monitored 125. The subsequent sample can comprise stabilized preserved nucleic acid from the subject. The monitoring can comprise detecting a presence or absence of CMV nucleic acid sequences in the subsequent sample by a high sensitivity test. In some embodiments, if CMV nucleic acids are present, the at-risk subject can be determined to have an active primary CMV infection 170. Optionally, confirmatory testing can be performed 180. Optionally, the subject determined to have an active primary CMV infection can be recommended for therapy for CMV infection 190, for example administration of antiviral compounds as described herein. When there is a presence of anti-CMV IgG and a presence of anti-CMV IgM 145, avidity testing 160 can be performed as described herein. Optionally, the avidity of the anti-CMV of the subject can be compared to that of a CMV developed control (or a threshold value) 165. If the avidity of the subject's IgG for CMV is comparable or greater than that of the CMV-developed control, the subject is determined to not have an active-primary CMV infection. If the avidity of the subject's IgG for CMV is neither comparable nor greater than that of the CMV-developed control or threshold value (e.g., weaker than that of the CMV-developed control or weaker than the threshold value), then the subject is determined to have an active primary active CMV infection 170. When there is a presence of IgG and an absence of IgM 140, the subject whose sample is received can be determined to not be at-risk for active primary CMV infection 155 (being determined to be “not at-risk” is not to say that active primary CMV infection is impossible for the subject, but merely that the subject is placed in a risk category in which active primary CMV infection is very unlikely, and thus there is unlikely to be a benefit to further testing the subject in accordance with some embodiments herein).

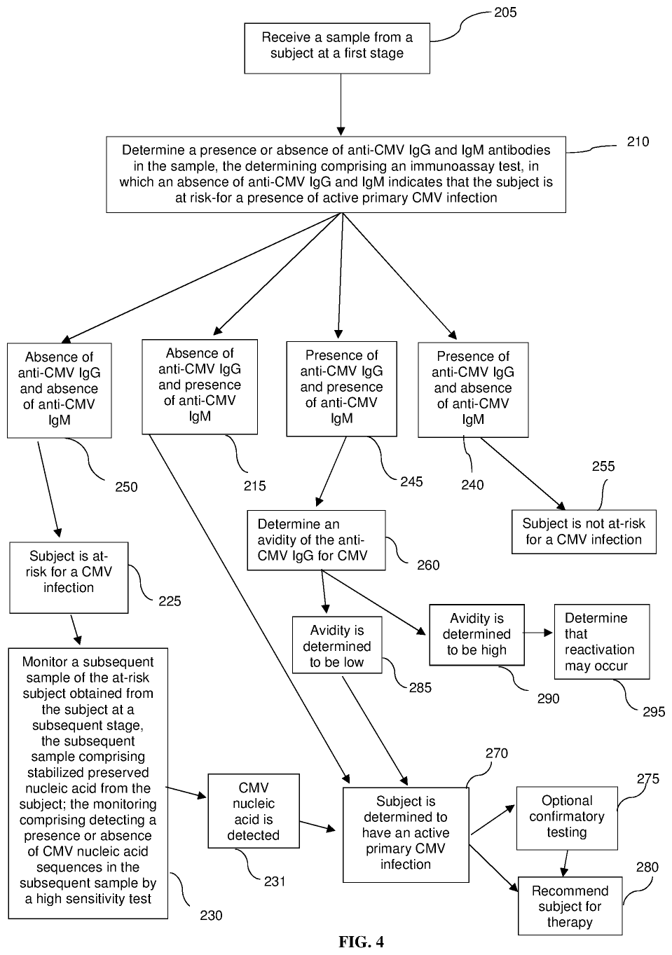

FIG. 4 is a flow diagram illustrating a method of determining a presence of active primary CMV infection according to some embodiments herein. In some embodiments, a sample of a subject (e.g., a blood sample) is received at a first stage 205. In some embodiments, a presence or absence of anti-CMV IgG and a presence or absence of anti-CMV IgM are determined 210. The presence or absence of the anti-CMV IgG can be determined using an immunoassay test as described herein, in which an absence of anti-CMV IgG indicates that the subject is at risk-for a presence of active primary CMV infection. When there is an absence of anti-CMV IgG and a presence of anti-CMV IgM 215, the subject can be determined to have an active primary CMV infection 270. After the subject is determined to have an active primary CMV infection 270, the subject may optionally undergo confirmatory—testing 275. Optionally, the subject determined to have an active primary CMV infection can be recommended for therapy for the CMV infection 280. When an absence of anti-CMV IgG and an absence of anti-CMV IgM are determined 250, the subject is at risk for a CMV infection (for example, a fetal-damaging CMV infection such as an active primary CMV infection) 225, and thus, of lower risk for having or developing a fetal-damaging CMV infection. If the subject is at-risk, a subsequent sample of the at-risk subject can be monitored 230. The subsequent sample can comprise stabilized preserved nucleic acid from the subject, for example in a blood or buccal sample. The monitoring can comprise detecting a presence or absence of CMV nucleic acid sequences in the subsequent sample by a high sensitivity test. If the CMV nucleic acid is present in the subsequent sample 231, the subject is determined to have an active primary CMV infection. 270. When a presence of anti-CMV IgG and a presence of anti-CMV IgM are determined 245, avidity testing 260 can be performed as described herein. The avidity can be determined to be low 285 or the avidity can be determined to be high 290. When the avidity is determined to be low 285, the subject can be determined to have an active primary CMV infection 270, and the subject can optionally undergo confirmatory orthogonal testing 275, and/or can be recommended for therapy for CMV infection as described herein 280. When the avidity is determined to be high 290, it is determined that reactivation may occur 295. When a presence of anti-CMV IgG and an absence of anti-CMV IgM 240 are determined, the subject can be determined to be not at-risk for an active primary CMV infection 255.

It is contemplated herein that a presence of anti-CMV IgM in a sample can be indicative of an active CMV infection. Accordingly, some embodiments of the method further comprise detecting a presence or absence of anti-CMV IgM in a sample of the subject. In some embodiments, the method of determining a presence of active primary CMV infection comprises determining a presence or absence of anti-CMV IgM in the sample (e.g., a sample) obtained from the subject at the first stage. If there is a presence of anti-CMV IgG and a presence of anti-CMV IgM in the sample, the method can further comprise determining an avidity of the anti-CMV IgG for CMV, for example by performing avidity testing as described herein. It is contemplated that a sample positive for anti-CMV IgM and for “high avidity” anti-CMV IgG can indicate a secondary and/or reactivation infection. “High avidity” anti-CMV IgG refers to comparable or higher avidity to CMV than IgG of a CMV-developed control; and/or higher avidity to CMV than IgG of a CMV-naïve control. It is contemplated that a sample positive for anti-CMV IgM and “low avidity” anti-CMV IgG can indicate an active primary CMV infection, rather than a secondary and/or reactivation infection. “Low avidity” anti-CMV IgG refers to lower avidity to CMV than IgG of a CMV-developed control, and/or comparable or lower avidity to CMV than IgG of a CMV-naïve control. A suitable CMV-developed control is understood to comprise anti-CMV IgG with high avidity to CMV. In some embodiments, a sample of the CMV-developed control is tested in the avidity testing as a control. The CMV-developed control sample can be of a comparable sample type to the sample of the subject (for example, both can be serum samples). In some embodiments, a stored value of the CMV-developed control is used as a reference in an avidity test, for example an electronically or optically stored value. In some embodiments, high avidity and/or low avidity is determined based on a comparison to a CMV-developed control and/CMV-naïve control sample. In some embodiments high avidity and/or low avidity is based on a comparison to an internal control. In some embodiments, high avidity is based on refers to an affinity constant that indicates tighter binding than a predetermined threshold, and/or “low avidity” is based on an affinity constant that indicates weaker binding than a predetermined threshold.

The method of determining a presence of active primary CMV infection can further comprise determining a presence or absence of anti-CMV IgM in the sample obtained from the subject at the first stage. If there is an absence of anti-CMV IgG and a presence of anti-CMV IgM in the sample, a near-term active, primary CMV infection is determined. In some embodiments, the method further comprises monitoring a subsequent sample. In some embodiments, an increase in levels of CMV nucleic acids over time indicates a presence of active primary CMV infection. In some embodiments, a level of CMV nucleic acids (which can indicate a “viral titer”) above a threshold (for example, a pre-determined value, or a level of a control) indicates a presence of active primary CMV infection. If the subject is determined to have active primary CMV infection, the method can further comprise confirmatory orthogonal testing.

Successive rounds of testing in accordance with methods, product combinations, and kits of some embodiments herein can increase the accuracy of detection of active primary CMV infection and conserve health care resources (by targeting high-sensitivity testing to a subset of subjects that are likely to benefit from it). The combination of immunoassay testing and high-sensitivity testing can further provide synergistic information on the stage of CMV infection (via the immunoassay testing for anti-CMV IgG and/or IgM), and can minimize the risk of false negatives and positives (via the high-sensitivity testing). Furthermore, by performing high-sensitivity tests on multiple subsequent samples as described herein, there is a lower likelihood of a false negative compared to a single high-sensitivity test (without being limited by theory, it is contemplated that a single test may not be performedat peak viral load level, and thus may miss the detection window, resulting in a lower sensitivity and a greater likelihood of a false negative). In some embodiments, the subject is pregnant (prenatal) and/or a transplant subject. In some embodiments, the blood sample comprises, consists essentially of, or consists of serum. In some embodiments, the blood sample comprises, consists essentially of, or consists of plasma.

In some embodiments, the subsequent sample is collected at a “subsequent stage,” that is at least 1 week after the first stage, for example at least 1, 2, 3, 4 or 5 weeks after the first stage, including ranges between any two of the listed values, such as 1-6, 1-4, 1-3, 1-2, 2-5, 2-4, 2-3, 3-5, 3-4, or 4-5 weeks after the first stage. In some embodiments, the subsequent stage is three weeks after the first stage. In some embodiments, monitoring the subsequent sample comprises high sensitivity tests on two or more subsequent samples that are obtained from the subject about 1, 2, 3, 4, 5, or 6 weeks apart from each other, including ranges between any two of the listed values, such as 1-6, 1-4, 1-3, 1-2, 2-5, 2-4, 2-3, 3-5, 3-4, or 4-5 weeks apart from each other.

The methods of embodiments herein can further be advantageous because they can minimize the need for the subject to return to a clinic, and thus can increase compliance by the subject, while conserving resources at the clinic. For example, in methods of some embodiments, the subsequent samples comprising nucleic acids are collected in the absence of a licensed health-care provider, such as at that subject's home. Stabilized nucleic acids can be sent from the subject to a testing site, which may be in a different location than where the samples were collected.

In some embodiments, if anti-CMV IgG is absent from the first-stage sample, and if CMV nucleic acid is present in the subsequent sample, the method further comprises confirmatory testing. Optionally, the confirmatory testing can be orthogonal. However, it is contemplated that in order to facilitate administering a CMV therapy to inhibit, reduce the likelihood or prevent transmission of CMV to the fetus (or to treat or inhibit CMV infection in utero) in accordance with methods and uses and product combinations of some embodiments herein, the confirmatory testing can be completed within 3, 2, or 1 weeks of determining that CMV nucleic acid is present in the subsequent sample. In some embodiments, the confirmatory testing comprises a second immunoassay test to confirm the presence or absence of anti-CMV IgG (and/or anti-CMV IgM) in the subsequent sample or another sample. Furthermore, if the second immunoassay test indicates the presence of both anti-CMV IgG and anti-CMV IgM, then the method can further comprise an avidity test as described herein to confirm the presence of active primary CMV infection. If there is a presence of anti-CMV IgM and an absence of anti-CMV IgG in the second immunoassay test, then the presence of a near-term primary CMV infection (a type of active primary CMV infection) can be confirmed. However, if there is an absence of anti-CMV IgG and an absence of anti-CMV IgM in the second immunoassay test, then the method can further comprise a second high-sensitivity test to determine the presence or absence of CMV nucleic acid sequences. The second high-sensitivity test can further comprise detecting consecutive increases in quantitative CMV nucleic acid measurements or CMV viral titer measurements. If both a first-stage and subsequent high-sensitivity test are positive for CMV, it is contemplated that the subject is very likely to have an active primary CMV infection. Additionally, if CMV nucleic acids increase in quantity over time, or if viral titer is above a specified threshold in the first-stage and subsequent test, the risk of fetal-damaging infection may be especially high. If CMV nucleic acid sequences are absent, or remain constant or decrease in quantity over time, an active primary CMV infection can be ruled-out. In some embodiments, the confirmatory orthogonal testing comprises detecting anti-CMV IgM after determining the presence of CMV nucleic acid.

The method of detecting active primary CMV infection can further comprise recommending to the subject that the first-stage sample be collected from the subject in a clinical setting and that the subsequent sample be collected in the absence of a phlebotomist, nurse, physician or other licensed health care professional (for example as an at-home test). In addition to recommending to the subject that a sample be collected in a clinical setting, the method can further comprise receiving a subsequent sample that was collected from the subject in a non-clinical setting and in the absence of the phlebotomist, nurse, physician or other licensed health care professional. The method can further comprise providing a sample receptacle to the subject. The sample receptacle can comprise reagents for stabilizing and preserving nucleic acids, such as buffers or buffer salts as described herein. The subject can provide the subsequent sample (for example, blood or saliva) to the sample receptacle The method can further comprise receiving the sample receptacle comprising the subsequent sample from the subject by carrier, such as mail courier, or the like.

The immunoassay test of any of the methods of determining a presence of active primary CMV infection of embodiments described herein can be selected from the group consisting of immunoprecipitation, particle immunoassays, immunonephelometry, radioimmunoassay, enzyme immunoassay, fluorescent immunoassay, or chemiluminescent immunoassay, or a combination of two or more of these. In some embodiments, the enzyme immunoassay comprises an enzyme-linked immunosorbent assay (ELISA).

The high sensitivity test of any of the methods described herein can be selected from the group consisting of polymerase chain reaction (PCR), sequencing, rolling circle amplification, or isothermal amplification (such as MDA). In some embodiments, the polymerase chain reaction comprises reverse transcription polymerase chain reaction (RT-PCR). In some embodiments, the polymerase chain reaction comprises quantitative polymerase chain reaction (qPCR), such as real-time PCR.

In some embodiments, a presence of high avidity anti-CMV IgG in the sample indicates a low likelihood of being at-risk subject for the transmission of CMV to a fetus. In such circumstances, it can be recommended that therapeutic intervention is not required. Optionally, if the subject has a low likelihood of being at risk, in methods, kits, and product combinations of some embodiments herein, a presence or absence of anti-CMV IgM is determined in a sample of the subject. If anti-CMV IgM is present in the sample, an avidity of the anti-CMV IgG for CMV can be determined as described herein, and it can be determined whether the subject has an active primary CMV infection.

In some embodiments, if a presence of an active, primary CMV infection is determined, the method further comprises recommending a therapy for CMV infection (which may also be referred to herein as a “CMV therapy”) for the subject, for example the administration of an antiviral compound, biologic, and/or immunotherapy as described herein. The antiviral compound can be selected from the group consisting of acyclovir, valacyclovir, ganciclovir, valganciclovir, foscarnet, cidofovir, cytogam, or a combination of two or more of these. In some embodiments, the antiviral compound is selected from the group consisting of, valacyclovir, ganciclovir, valganciclovir, foscarnet, cidofovir, or a combination of two or more of these. In some embodiments the therapy is recommended to be performed within the first half of pregnancy, for example in the first trimester, or in the first trimester or first half of the second trimester. In some embodiments, the recommendation of therapy is given within 1, 2, 3, 4, or 5 weeks of detection of infection (including ranges between any two of the listed values, for example within 1-5 weeks, 1-4 weeks, 1-3, weeks, 1-2 weeks, 2-5 weeks, 2-4 weeks, 2-3 weeks, 3-5 weeks, 3-4 weeks, or 4-5 weeks). In some embodiments, it is recommended that the therapy takes place during the first half of pregnancy of the subject, and/or prior to transmission to a fetus or appearance of symptoms of a fetus. In some embodiments, the decision for therapy can be based on consecutive increases in levels of CMV nucleic acids in samples of the patient, as determined by high-sensitivity testing as described herein. In some embodiments, the method comprises administering the recommended therapy.

### Methods of Treating CMV Infection

In some embodiments, a method of treating, inhibiting, or ameliorating CMV infection or a symptom thereof is described. The method can comprise receiving a result of an immunoassay test as described herein to determine a presence or absence of anti-CMV IgG in sample obtained from the subject (e.g., a blood sample). If anti-CMV IgG is absent from the sample, the method can further include receiving results of a high sensitivity test to determine a presence or absence of CMV nucleic acid sequences of the subject. When anti-CMV IgG is absent and CMV nucleic acid sequences are present, the subject can be determined to have an active, primary CMV infection. The method can further comprise administering a therapy for the CMV infection in the subject (or a fetus of the subject) determined to have an active, primary CMV infection. By way of non-limiting example, the therapy can comprise, an art-recognized therapy for CMV infection. In some embodiments, the therapy for CMV infection comprises an antiviral compound as described herein (e.g., acyclovir, valacyclovir, ganciclovir, valganciclovir, foscarnet, cidofovir, cytogam, or a combination of two or more of these), a biologic (such as a monoclonal antibody specific for CMV), an immunotherapy such as genetically modified immune cell (such as CAR-T). It is noted that some immunotherapies may also comprise, consist essentially of, or consist of biologics. In some embodiments, the immunotherapy comprises a cell therapy such as genetically modified T cells and/or B cells. In some embodiments, the antiviral compound is administered orally. In some embodiments, the therapy for the CMV infection comprises terminating the pregnancy of the subject.

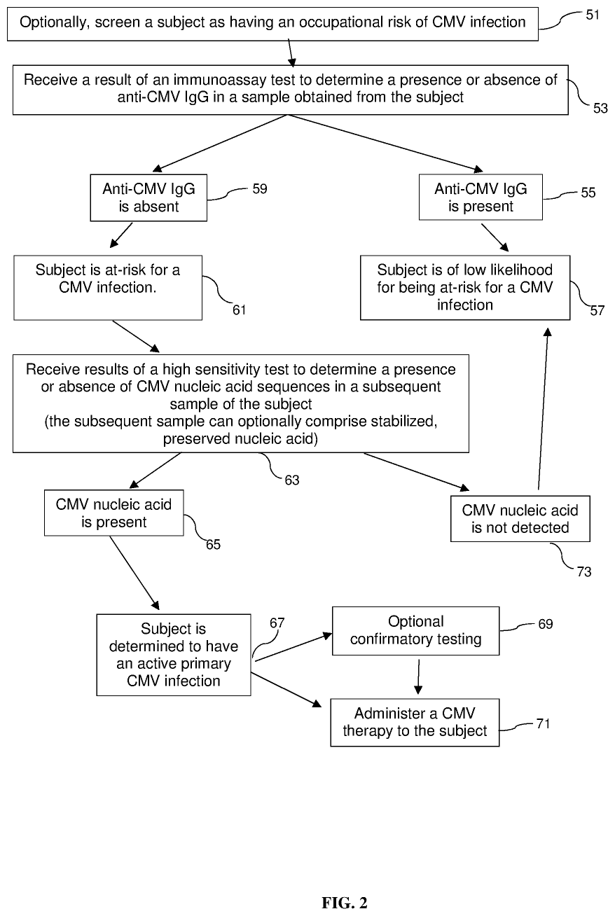

FIG. 2 is a flow diagram illustrating a method of treating, inhibiting, or ameliorating CMV infection or a symptom thereof in accordance with some embodiments. Optionally, the method can include screening the subject as having an occupational risk of CMV infection 51. Examples of occupational risks of CMV infection include a nurse, elementary or middle school teacher, nursing or psychiatric or home health aide, child care worker, teacher assistant, or primary child care provider for a preschooler. The method can include receiving a result of an immunoassay test to determine a presence or absence of anti-CMV IgG in a sample obtained from the subject 53. By way of example, the immunoassay test can be performed on a sample comprising serum, plasma, or (whole) blood of the subject. An absence of anti-CMV IgG can indicate that the subject is at-risk for a CMV infection (for example an active primary CMV infection), such as a fetal-damaging CMV infection. If anti-CMV IgG is present 55, the subject can be of low likelihood for being at-risk for a CMV infection 57 (for example active primary infection), and unlikely to have or develop a fetal-damaging CMV infection. If anti-CMV IgG is absent 59, the subject can be at-risk for active primary CMV infection 61. Accordingly, the method can comprise receiving results of a high sensitivity test to determine a presence or absence of CMV nucleic acid sequences in a subsequent sample of the subject 63. Optionally, the high-sensitivity tests can have been performed on a nucleic-acid-containing subsequent sample of the subject collected in a non-clinical setting (“at-home”). The subsequent sample can optionally comprise stabilized, preserved nucleic acid. The subsequent stage can be at least 1 week after the first stage, for example at least 1, 2, 3, 4 or 5 weeks after the first stage, including ranges between any two of the listed values as described herein. If CMV nucleic acid is present (as detected by the high-sensitivity test) 65, the subject can be determined to have an active, primary CMV infection 69. Accordingly, a CMV therapy can be administered to the subject 71. Example CMV therapies include an antiviral compound, a biologic, an immunotherapy, and termination of a pregnancy, or a combination of two or more of the listed items. By way of example, the antiviral compound can be selected from the group consisting of acyclovir, valacyclovir, ganciclovir, valganciclovir, foscarnet, cidofovir, and cytogam, or can comprise a combination of two or of the listed items. It is contemplated that valacyclovir has a longer half-life than many other antiviral compounds (such as acyclovir). Accordingly, the antiviral compound can comprise, consist essentially of, or consist of valacyclovir. Optionally, confirmatory testing can be performed 69 prior to administering the CMV therapy. It is contemplated that the CMV therapy can be useful to inhibit, prevent, or reduce the likelihood of CMV transmission to a fetus (for example, in the case of a pregnant subject), and/or to treat or inhibit CMV infection in utero. Accordingly, confirmatory testing can be completed within 3, 2, or 1 week so as to facilitate intervention with CMV therapy prior to transmission to the fetus. Thus, the CMV therapy can be administered to the subject 71 within 3, 2, or 1 week of determining CMV nucleic acid to be present in the subsequent sample 65. Thus, the method can comprise a prophylactic dose of the CMV therapy. The method can be for inhibiting, reducing the likelihood, or preventing transmission of CMV to the fetus of the pregnant subject, and/or treating or inhibiting CMV infection in utero. If CMV nucleic acid is not detected 73, the subject can be of low likelihood for being at-risk for active primary CMV infection 59. In some embodiments, for any of the methods described herein, the CMV nucleic acid is present 65, and as such, the subject is determined to have an active primary CMV infection 67.

In some embodiments, the method of treating, inhibiting, or ameliorating CMV infection or a symptom thereof comprises receiving the results of any method of determining a presence of active primary CMV infection as described herein. If the subject has an active primary CMV infection, the method can further comprise administering a therapy for CMV infection as described herein. Optionally, the decision to administer the therapy can be based on consecutive increases in levels of CMV nucleic acids in samples of the patient, as determined by high sensitivity testing as described herein.

In some embodiments, if the subject is pregnant, the therapy for the CMV infection (such as an antiviral compound as described herein) is administered to treat the fetus in utero. In some embodiments, the therapy is administered in the first half of the pregnancy (for example, in the first trimester, and/or in the first half of the second trimester). In some embodiments, the therapy is administered within 1, 2, 3, 4, 5, or 6 weeks of infection, including ranges between any two of the listed values, for example, 1-6, 2-5, 1-3, or 2-3. In some embodiments, the therapy is administered within three weeks of infection. In some embodiments, the therapy is administered within one to two weeks of infection. In some embodiments, the therapy is administered prior to transmission of CMV to a fetus or the appearance of CMV infection symptoms in the fetus. As such, in some embodiments, the therapy is administered within 3, 2, or 1 weeks after CMV nucleic acids are determined to be present in the subsequent sample of the subject. Example protocols for treating a fetus in utero with an antiviral compound are described in Jacquemard et al., BJOG 2007 114: 1113, which is hereby incorporated by reference in its entirety. While the study of Jacquemard et al. uses valaciclovir, it is contemplated that in some embodiments, the method of treating, inhibiting, or ameliorating CMV infection or a symptom thereof uses a similar protocol as Jacquemard et al., but for an antiviral compound other than valaciclovir, as described herein. In some embodiments, the method of treating, inhibiting, or ameliorating CMV infection or a symptom thereof uses a similar protocol as Jacquemard et al., and the antiviral compound comprises, consists essentially of, or consists of valaciclovir. In some embodiments, the method of treating CMV infection comprises terminating the pregnancy of the subject. In some embodiments, the method inhibits, treats, or ameliorates CMV infection in a fetus in utero. In some embodiments, the method inhibits, reduces the likelihood, or prevents transmission of CMV from the subject to a fetus of the subject. By way of example, transmission of CMV to a fetus can be measured by viremia in the baby (after being born).

In some embodiments, the therapy for CMV infection comprises an antiviral compound selected from the group consisting of acyclovir, valacyclovir, ganciclovir, valganciclovir, foscarnet, cidofovir, cytogam, or a combination of any two of these. In some embodiments, the therapy for CMV infection comprises an antiviral compound selected from the group consisting of valacyclovir, valaciclovir, ganciclovir, valganciclovir, foscarnet, cidofovir, or a combination of any two of these. In some embodiments, an additional therapeutic compound is administered with the antiviral compound. The antiviral compound can be administered via any suitable route of administration, for example, orally, intravenously, intraperitoneally, subcutaneously, via inhalation, or topically. In some embodiments, the antiviral compound does not comprise valaciclovir. In some embodiments, the treatment for CMV infection, when administered, is effective to treat, inhibit, or ameliorate CMV infection. In some embodiments the CMV therapy comprises an antiviral compound at a prophylactic dose that is lower than a lowest dose of the antiviral compound approved by a regulatory authority (e.g., the FDA or EMA) for CMV viremia.

In some embodiments, additional high-sensitivity testing for CMV nucleic acids is performed in the course of therapy for CMV infection. The amount of CMV therapy (e.g., amount of antiviral compound, biologic, and/or immunotherapy) can be adjusted based on the total level of CMV nucleic acid in the sample or the rate of change in CMV nucleic acids levels between successive tests.

### Product Combinations

Some embodiments include kits and/or product combinations useful in methods of detecting a presence or absence of active primary CMV infection. Kits and product combinations as described herein can comprise reagents useful for methods of determining a presence or absence of active primary CMV infection, and/or methods of treating CMV infection. An “immunoassay kit” or “immunoassay test kit” as used herein comprises reagents for performing an immunoassay test to detect anti-CMV IgG, and optionally anti-CMV IgM as described herein. For example, an immunoassay kit of some embodiments can comprise CMV antigen, and/or antibody specific for human IgG, and/or antibody specific for human IgM. Optionally an immunoassay kit comprises additional reagents for immunoassay tests as described herein, for example labels, detection enzymes, buffers, multi-well plates, and the like. A “high-sensitivity kit” or “high-sensitivity test kit” as used herein comprises reagents for high-sensitivity nucleic acid test as described herein. For example, a high-sensitivity kit of some embodiments comprises primers or probes specific for CMV nucleic acids as described herein. Optionally, a high-sensitivity kit comprises additional nucleic acid testing reagents, for example polymerase, reverse transcriptase, buffers, labels, and the like. As used herein, a “product combination” refers to a product comprising two or more components, which can be provided together or separately. For example, a product combination in accordance with embodiments herein can comprise an immunoassay kit and a high-sensitivity kit in a single container or package, or it can comprise an immunoassay kit and a high-sensitivity kit in separate containers or packages. The components of a product combination are not necessarily shipped in the same shipment, though they may be.

In some embodiments, a product combination for determining a presence or absence of active primary CMV infection in a subject comprises, consists essentially of, or consists of an immunoassay kit and a high-sensitivity kit. In some embodiments, the immunoassay kit comprises a protein that binds specifically to anti-CMV IgG, for example CMV antigen and/or anti-human IgG. In some embodiments, the anti-human IgG is specific for human anti-CMV IgG. In some embodiments, the high sensitivity kit comprises nucleic acid that hybridizes to CMV-specific nucleic acid sequences, e.g., stabilized preserved nucleic acids. In some embodiments, the immunoassay kit is for use at a clinic. In some embodiments, the immunoassay kit comprises instructions indicating licensed medical professionals are to collect samples to test for a presence or absence of anti-CMV IgG.

In some embodiments, the high-sensitivity kit comprises, consists essentially of, or consists of reagents for use at the subject's home, for example a sample receptacle. In some embodiments, the high-sensitivity kit is not used to collect a sample at a clinic. In some embodiments, the high sensitivity kit is for use at a clinic. In some embodiments, the high sensitivity kit comprises a sample receptacle for collection (e.g., non-clinical collection such as home collection) of stabilized preserved nucleic acid sequences from the subject. The sample receptacle can comprise reagents for stabilizing and preserving nucleic acids, such as buffers or buffer salts, for example EDTA. In some embodiments, the high sensitivity kit comprises instructions for sending stabilized preserved nucleic acid obtained from the subject to a laboratory for testing. In some embodiments, the immunoassay kit is for use at a clinic.

In some embodiments, the product combination comprises a first immunoassay kit, a high sensitivity kit, and a second immunoassay kit. In some embodiments, the second immunoassay kit is used to determine the presence or absence of anti-CMV IgG and anti-CMV IgM. In some embodiments, the second immunoassay kit comprises a protein that binds specifically to anti-CMV IgG, for example CMV antigen and/or an antibody specific for human IgG. In some embodiments, the second immunoassay kit further comprises a protein that binds specifically to anti-CMV IgM, for example CMV antigen and/or an antibody specific for human IgM. In some embodiments, the product combination further comprises instructions indicating the second immunoassay kit is to be used for confirmatory testing, for example if the first immunoassay kit and the high-sensitivity kit indicate a presence of active primary CMV infection as described herein.

In some embodiments, the product combination further comprises an avidity test kit (comprising reagents for avidity testing as described herein). For example, the kit can comprise an ELISA for CMV IgG and a chaotropic ion. The avidity test kit can be used to measure an avidity of anti-CMV IgG of a subject for CMV as described herein. In some embodiments, the avidity test kit is used to confirm the presence or absence of CMV. In some embodiments, the product combination comprises instructions for using such an avidity test kit indicating that it is to be used up to about three weeks after the second immunoassay kit.

In some embodiments, the product combination comprises an immunoassay kit comprising one or more antibodies that bind specifically to human IgG or IgM. The product combination can further comprise a high-sensitivity kit as described herein. The immunoassay kit can further comprise a detectable moiety. Optionally, the detectable moiety is bound to the antibody specific for human IgG or IgM, or to a secondary antibody that specifically binds to the antibodies specific for human IgG or IgM. By way of example, such immunoassay kits can be useful for diagnosing or detecting CMV. The immunoassay kit of the product combination can comprise reagents for any of a variety of immunoassays, for example, ELISA, western blot, later flow assays, no-wash assays, and the like, and can include reagents for any of these assays. The immunoassay kit of the product combination can comprise at least one of IgG or IgM, for example as a positive control. Optionally, the immunoassay kit comprises an IgG or IgM that binds to CMV. The kits can comprise one or more detectable moieties. Example detectable moieties include fluorophores, radiolabels, FRET pairs, enzyme-substrate pairs, enzymes, and the like as described herein.

The product combination of some embodiments furthers comprises positive and/or negative controls. An example of a positive control is a sample, for example a blood sample, known to comprise CMV nucleic acids, and/or known to comprise anti-CMV IgG and/or anti-CMV IgM. An example of a negative control is a sample (e.g., a biological sample) that does not comprise CMV.

An immunoassay kit of the product combination of some embodiments can further comprise, if desired, one or more of additional components, for example, containers with one or more buffers (e.g., wash buffers), detection reagents, or antibodies. Printed instructions, either as inserts or as labels, indicating quantities of the components to be used and guidelines for their use, can also be included in the kit. In the present disclosure it will be understood that the specified materials and conditions can be useful in accordance with some embodiments herein, but that unspecified materials and conditions are not excluded so long as they do not prevent the immunoassay kit from suitably detecting markers of CMV infection, such as anti-CMV IgG.

In some embodiments, the immunoassay kit of the product combination includes reagents for carrying out ELISAs (e.g., multi-well plates, 96-well plates; plates containing wells in multiples of 96, and the like). In some embodiments, the kit includes substrates for lateral flow assays. In some embodiments, the kit includes reagents for western blot. In some embodiments, the kits includes components for conducting immunohistochemical analysis of a tissue sample (e.g. fixative, slides, and the like).

In addition to above-mentioned components, the subject kits of the product combination of some embodiments can further include instructions for using the components of the kit in accordance with methods described herein. The instructions for using the kits of the product combination in accordance with methods described herein are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In some embodiments, the instructions are present as an electronic storage data file present on a suitable computer readable storage medium, e.g. flash drive, DVD-ROM, CD-ROM, and the like. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Additional Embodiments

Some embodiments screen for subjects who are at risk of CMV infection during pregnancy and subsequently, based on the subjects' risk, monitor them to determine if a subject has become infected and whether the infection is active and the general level of viral titer. This information can allow for critical clinical decision making resulting improved subject outcomes and the development of novel treatment paradigms.

Some embodiments comprise a clinical management protocol, comprising testing all subjects for the presence of anti-CMV IgG in samples obtained from such subjects via immunological assay. If there is an absence of anti-CMV IgG, the subject is considered to be at a high risk (or “at-risk”) for a CMV infection. High risk subjects (e.g., subjects who are at-risk for active primary CMV infection) would then be subsequently monitored on a frequent basis by detecting for a presence or absence of nucleic acid sequences specific to CMV from samples collected in the comfort of their own homes. The sample collection can be performed by a home blood collection device that allows for the stabilization and preservation of RNA and DNA that are specific to CMV. These samples would be sent directly to a lab for testing of the presence of nucleic acid sequences specific to CMV. Subsequent confirmatory and orthogonal testing can be performed to confirm whether the subject has a primary, active CMV infection. If there is a presence of anti-CMV IgG in the sample of the subject, the subject is considered to have a low likelihood of being at-risk for a CMV infection. As used herein, “low likelihood” of being at-risk for active primary CMV infection refers to a risk category in which active primary CMV has not been ruled-out, but based on information obtained thus far, for example a presence of anti-CMV IgG, the subject has a lower likelihood of having an primary CMV infection than a comparable untested subject. By way of example, a comparable untested subject for a pregnant female can be another pregnant female of the general population who has not been tested for CMV infection. A subject having a “low likelihood” of being at-risk for active primary CMV infection in accordance with methods, kits, and product combinations of some embodiments herein has a greater probability of being at-risk for active primary CMV infection than a subject whose sample(s) exhibit a presence of anti-CMV IgG and an absence of anti-CMV IgM; but a lower probability of being at-risk for active primary CMV infection than a subject whose sample(s) exhibit an absence of anti-CMV IgG. Optionally, for a subject who has a low likelihood of active primary CMV infection (a “low likelihood subject”), the sample (or another sample of the subject) can further be tested for a presence or absence of anti-CMV IgM. If the sample is determined to have an absence of anti-CMV IgM, the low likelihood subject is not at-risk for active primary CMV infection. If the sample is determined to have a presence of anti-CMV IgM, the risk of active primary CMV infection remains, and avidity testing can be performed as described herein to provide additional information on whether the subject can be determined to have an active primary CMV infection.

Overall, the methods of some embodiments have a number of advantages over conventional clinical management paradigms. They can allow for a non-invasive and economical approach to CMV testing during pregnancy, which is not available with existing testing approaches. The can provide timely and actionable information for further invasive diagnostic testing only in those subjects which should be subjected to such higher risk, invasive testing. They can allow for the rapid detection and intervention of CMV infections that cause the most newborn and childhood disease, and therefore can reduce their impact on society. In some embodiments, an outcome or therapy after the detection of a primary infection comprises termination of the pregnancy.

The following embodiments are described herein:

1. A method of determining a presence of active primary cytomegalovirus (CMV) infection, the method comprising:
receiving a sample obtained from a subject at a first stage;
determining a presence or absence of anti-CMV IgG in the sample, said determining comprising an immunoassay test, wherein an absence of anti-CMV IgG indicates that the subject is at-risk for active primary CMV infection;
if the subject is at-risk, monitoring a subsequent sample of the subject obtained from the subject at a subsequent stage,
the subsequent sample comprising nucleic acid of the subject;
said monitoring comprising detecting a presence or absence of CMV nucleic acid sequences in the subsequent sample by a high sensitivity test,
wherein an absence of anti-CMV IgG in the sample and a presence of CMV nucleic acid in the subsequent sample indicate a presence of active primary CMV infection.

2. The method of embodiment 1, further comprising determining a presence or absence of anti-CMV IgM in the sample obtained from the subject at the first stage,

wherein if there is a presence of anti-CMV IgG and a presence of anti-CMV IgM in the sample, the method further comprises determining an avidity of the anti-CMV IgG for CMV, and

wherein the anti-CMV IgG having a comparable or higher avidity to CMV than IgG of a CMV-developed control indicates that the subject has an active primary CMV infection.

3. The method of embodiment 1, further comprising determining a presence or absence of anti-CMV IgM in the sample obtained from the subject at the first stage,

wherein if there is an absence of anti-CMV IgG and a presence of anti-CMV IgM in the sample, a near-term active primary CMV infection is determined, and the method further comprises said monitoring of the subject.

4. The method of Embodiment 3, further comprising confirmatory testing.

5. The method of any one of Embodiments 1-4, wherein the subject is prenatal and/or a transplant subject.

6. The method of any one of Embodiments 1-5, wherein the sample comprises serum.

7. The method of any one of Embodiments 1-5, wherein the sample comprises plasma.

8. The method of any one of Embodiments 1-7, wherein the subsequent stage is at least three weeks after the first stage.

9. The method of any one of Embodiments 1-8, wherein monitoring the subsequent sample comprises high sensitivity tests on samples that were obtained from the subject three weeks apart from each other.

10. The method of any one of Embodiments 1-9, wherein if anti-CMV IgG is absent from the sample, and if CMV nucleic acid is present in the subsequent sample, the method further comprises confirmatory orthogonal testing.

11. The method of any one of Embodiment 10, wherein the confirmatory orthogonal testing comprises a second immunoassay test to confirm a presence or absence of anti-CMV IgG and anti-CMV IgM in the subsequent sample or another sample.

12. The method of Embodiment 11, wherein if the second immunoassay test indicates the presence of both anti-CMV IgG and anti-CMV IgM, then the confirmatory orthogonal testing further comprises an avidity test to determine IgG avidity for CMV.

13. The method of Embodiment 11, wherein a presence of anti-CMV IgM and an absence of anti-CMV IgG in the second immunoassay test confirm the presence of active primary CMV infection.

14. The method of Embodiment 11, wherein if both anti-CMV IgG and anti-CMV IgM are absent in the second immunoassay test, then the method further comprises a second high sensitivity test to determine a presence or absence of CMV nucleic acid sequences in the subsequent sample or another sample.

15. The method of Embodiment 14, wherein the second high sensitivity test comprises detecting consecutive increases in quantitative CMV nucleic acid measurements or in CMV viral titer measurements.

16. The method of Embodiment 11, wherein the confirmatory orthogonal testing comprises detecting anti-CMV IgM after determining the presence of the CMV nucleic acid.

17. The method of any one of Embodiments 1-16, further comprising recommending to the subject that the sample be collected in a clinical setting and that the subsequent sample be collected in the absence of a phlebotomist, nurse, physician or other licensed health care professional.

18. The method of Embodiment 17, further comprising collecting the subsequent sample in a non-clinical setting in the absence of the phlebotomist, nurse, physician or other licensed health care professional.

19. The method of any one of Embodiments 1-18, wherein the immunoassay test is selected from the group consisting of: immunoprecipitation, particle immunoassays, immunonephelometry, radioimmunoassay, enzyme immunoassay, fluorescent immunoassay, and chemiluminescent immunoassay.

20. The method of Embodiment 19, wherein the enzyme immunoassay comprises an enzyme-linked immunosorbent assay (ELISA).

21. The method of any one of Embodiments 1-20, wherein the high-sensitivity test is selected from the group consisting of polymerase chain reaction, sequencing, and rolling amplification.

22. The method of Embodiment 21, wherein the polymerase chain reaction comprises reverse transcription polymerase chain reaction (RT-PCR).

23. The method of any one of Embodiments 1-22, wherein the presence of high avidity anti-CMV IgG in the sample indicates a low risk subject for the transmission of CMV to a fetus.

24. The method of any one of Embodiments 1-22, wherein if a presence of an active, primary CMV infection is indicated, the method further comprises recommending a therapy for CMV infection for the subject.

25. The method of Embodiment 24, wherein the therapy for CMV infection is selected from the group consisting of: an antiviral compound, a biologic, an immunotherapy, or termination of a pregnancy.

26. The method of Embodiment 24, wherein the antiviral compound is selected from the group consisting of: valacyclovir, ganciclovir, valganciclovir, foscarnet, and cidofovir.

27. The method of any one of Embodiments 24-26, wherein the therapy is recommended within three weeks of detection of infection, during the first half of the pregnancy of the subject, and/or prior to transmission to a fetus or appearance of symptoms in a fetus.

28. A method of treating cytomegalovirus (CMV) infection in a subject, the method comprising:
receiving a result of an immunoassay test to determine a presence or absence of anti-CMV IgG in a sample obtained from the subject;
if anti-CMV IgG is absent from the sample, receiving results of a high sensitivity test to determine a presence or absence of CMV nucleic acid sequences in a subsequent sample of the subject;
determining the subject to have an active primary CMV infection when anti-CMV IgG is absent and CMV nucleic acid is detected; and
administering a CMV therapy to the subject determined to have an active, primary CMV infection.

29. The method of Embodiment 28, wherein the result of the immunoassay test also determines a presence or absence of anti-CMV IgM in the sample obtained from the subject.

30. The method of Embodiment 29, wherein if anti-CMV IgM is absent from the sample, the method further comprises receiving results of a high sensitivity test to determine a presence or absence of stabilized preserved CMV nucleic acid sequences from the subject.

31. The method of Embodiment 28-30, wherein determining the subject to have an active, primary CMV infection comprises receiving at least one of:

a measurement of total CMV nucleic acid in the subsequent sample of the subject, or

a measurement of a rate of change in CMV nucleic acid levels in the subject.

32. The method of any one of Embodiments 28-31, wherein the subject is pregnant, and wherein the antiviral compound is administered to treat a fetus of the subject in utero.

33. The method of Embodiment 32, wherein the subject is treated in the first half of pregnancy, within three weeks of infection, and/or prior to transmission of CMV to a fetus or appearance of CMV infection symptoms in the fetus.

34. The method of any one of Embodiments 28-33, wherein the CMV therapy is selected from the group consisting of an antiviral compound, a biologic, an immunotherapy, and termination of a pregnancy.

35. The method of any one of Embodiment 34, wherein the antiviral compound is selected from the group consisting of valacyclovir, valaciclovir, ganciclovir, valganciclovir, foscarnet, and cidofovir.

36. The method of any one of Embodiments 28-35, further comprising receiving a result of an immunoassay test to determine a presence or absence of anti-CMV IgM in the sample,
wherein if there is a presence of anti-CMV IgG and anti-CMV IgM in the sample, the method further comprises determining an avidity of the anti-CMV IgG for CMV, and
wherein the anti-CMV IgG having a comparable or lower avidity to CMV than a CMV-naïve control indicates that the subject is at-risk for an active, primary CMV infection.

37. The method of any one of Embodiments 28-36, further comprising receiving the results of an immunoassay test to determine a presence or absence of anti-CMV IgM in the sample,
wherein a presence of anti-CMV IgG and an absence of anti-CMV IgM indicates a prior infection and an absence of an active, primary infection, meaning the subject is low-risk.

38. The method of any one of Embodiments 28-37, wherein the presence of high avidity anti-CMV IgG indicates a low risk of active, primary CMV infection.

39. The method of any one of Embodiments 28-38, wherein if the CMV nucleic acid sequences are detected, the method further comprises receiving a result of an immunoassay test for anti-CMV IgG and anti-CMV IgM after the high sensitivity test.

40. The method of Embodiment 39, wherein if both anti-CMV IgG and anti-CMV IgM are present, the method further comprises receiving a result of an avidity test to confirm the presence of CMV.

41. The method of Embodiment 39, wherein if anti-CMV IgM is present and anti-CMV IgG is absent, the subject is determined to have an active primary CMV infection.

42. The method of Embodiment 39, wherein if both anti-CMV IgG and anti-CMV IgM are absent, the method further comprises receiving the results of a subsequent high-sensitivity test to determine the presence or absence of CMV nucleic acid sequences.

43. The method of Embodiment 42, wherein the subsequent high-sensitivity test comprises detecting consecutive increases in quantitative CMV nucleic acid measurements and/or consecutive increases CMV viral titer measurements, indicating an active primary CMV infection.

44. The method of any one of Embodiments 1-43, wherein the sample comprises a blood sample, such as whole blood, serum, or plasma.

45. The method of any one of Embodiments 1-44, wherein the subsequent sample comprises stabilized preserved nucleic acids of the subject.

46. A product combination for determining the presence of cytomegalovirus (CMV) in a subject, the product combination comprising:

an immunoassay kit comprising a protein that binds specifically to anti-CMV IgG; and

a high sensitivity kit comprising nucleic acid that specifically hybridizes to a stabilized preserved CMV nucleic acid.

47. The product combination of Embodiment 46, wherein the immunoassay kit further comprises a protein that binds specifically to anti-CMV IgM.

48. The product combination of Embodiment 46 or Embodiment 47, wherein the high sensitivity kit comprises a receptacle for collection of stabilized preserved nucleic acid from the subject.

49. The product combination of any one of Embodiments 46-47, wherein the high sensitivity kit comprises instructions for sending stabilized preserved nucleic acid obtained from the subject to a laboratory for testing.

50. The product combination of any one of Embodiments 46-49, wherein the immunoassay kit is for use at a clinic.

51. The product combination of any one of Embodiments 46-50, further comprising a second immunoassay kit for determining a presence or absence of anti-CMV IgG and anti-CMV IgM, the second immunoassay kit comprising:
a protein that binds specifically to anti-CMV IgG; and
a protein that binds specifically to anti-CMV IgM.

52. The product combination of Embodiment 51, further comprising instructions to use the second immunoassay kit up to three weeks after the high-sensitivity kit.

53. The product combination of any one of Embodiments 46-52 further comprising an avidity test kit.

54. The product combination of Embodiment 53, further comprising instructions for using the avidity test kit up to three weeks after the second immunoassay kit.

### Example 1

A blood sample is collected from a subject at a first stage, during the first trimester of pregnancy. The blood sample is collected in a clinic. An immunoassay test comprising sandwich ELISA is performed, and determine that anti-CMV IgG is absent from the subject's blood sample. Sandwich ELISA is further performed on the sample, and determines that anti-CMV IgM is absent from the subject's blood sample. The subject is determined to be at-risk for an active primary CMV infection.

Subsequently, blood samples are obtained from the subject at subsequent stages three and six weeks after the first stage, and these subsequent blood samples are monitored by high-sensitivity testing. The high-sensitivity testing comprises quantitative PCR for CMV nucleic acids. The first subsequent blood sample (obtained from the subject three weeks after the first-stage sample) is obtained from the subject at the subject's home. The quantitative PCR indicates is a presence of CMV nucleic acid in the first subsequent sample. The second subsequent blood sample is obtained from the subject at the subject's home three weeks after the first subsequent blood sample. Quantitative PCR is performed on the second subsequent blood sample. The second subsequent blood sample has quantitatively more CMV nucleic acid (indicating an increased CMV viral titer compared to the first subsequent blood sample). The subject is determined to have an active primary CMV infection. The subject is recommended for therapy for active primary CMV infection with valaciclovir so that the fetus can be treated as well. The therapy is recommended before completing the first half of the second trimester.

### Example 2

A blood sample is obtained from a subject at a doctor's office. A no-wash immunoassay is performed on the blood sample, and determines that anti-CMV IgG is present in the sample. A no-wash immunoassay is also used to determine that anti-CMV IgM is absent from the sample. The subject is determined to be not at-risk for an active primary CMV infection.

### Example 3

A subject is pre-screened as having an occupational risk of CMV infection. In particular, the subject is a pregnant female identified as a preschool teacher. A sample of blood is collected from a subject at a first stage, during the first trimester of pregnancy. The sample is collected in a clinic. An immunoassay test comprising a lateral flow immunoassay is performed, and it is determined that anti-CMV IgG is absent from the subject's blood sample. The subject is determined to be at-risk for an active primary CMV infection.

Subsequently, saliva samples are obtained from the subject at subsequent stages two and four weeks after the first stage, and these subsequent saliva samples are monitored by high-sensitivity testing (it is noted that urine samples would also be acceptable subsequent samples). The high-sensitivity testing comprises quantitative PCR for CMV nucleic acids. The first subsequent saliva sample (obtained from the subject two weeks after the first-stage sample) is obtained from the subject at the subject's home. The quantitative PCR indicates is a presence of CMV nucleic acid in the first subsequent sample. The second subsequent saliva sample is obtained from the subject at the subject's home three weeks after the first subsequent saliva sample. Quantitative PCR is performed on the second subsequent saliva sample. The second subsequent saliva sample has quantitatively more CMV nucleic acid (indicating an increased CMV viral titer compared to the first subsequent saliva sample). The subject is determined to have an active primary CMV infection. The subject is recommended for therapy with valacyclovir at a prophylactic dose, for the purpose of inhibiting transmission of CMV to the fetus. The therapy is recommended before completing the first half of the second trimester.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a method having at least one of A, B, and C” would include but not be limited to method that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a method having at least one of A, B, or C” would include but not be limited to method that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Wherever a method of using a composition or product combination or kit (e.g., a method of using an immunoassay test and a high-sensitivity test) is disclosed herein, the corresponding product combination or kit for use is also expressly contemplated.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1.-76. (canceled)

77. A method of determining a presence of active primary cytomegalovirus (CMV) infection, the method comprising:
receiving a sample obtained from a pregnant subject at a first stage;
determining a presence or absence of anti-CMV IgG in the sample, said determining comprising an immunoassay test, wherein an absence of anti-CMV IgG indicates that the subject is at-risk for CMV infection;
if the subject is at-risk, monitoring a subsequent sample of the subject obtained from the subject at a subsequent stage,
the subsequent sample comprising nucleic acid of the subject,
said monitoring comprising detecting a presence or absence of CMV nucleic acid sequences in the subsequent sample by a high sensitivity test,
wherein an absence of anti-CMV IgG in the sample and a presence of CMV nucleic acid in the subsequent sample indicate a presence of active primary CMV infection.

78. The method of claim 77, further comprising recommending CMV therapy to the subject if the monitoring detects a presence of CMV nucleic acids in the subsequent sample.

79. The method of claim 78, wherein the CMV therapy comprises an antiviral compound selected from the group consisting of: acyclovir, valacyclovir, ganciclovir, valganciclovir, foscarnet, cytogam, and cidofovir.

80. The method of claim 78, wherein the CMV therapy is recommended within three weeks of detection of infection, and wherein the therapy is recommended for administration in the first trimester of pregnancy or the first half of the second trimester of pregnancy.

81. The method of claim 77, wherein the subsequent sample comprises stabilized, preserved CMV nucleic acid.

82. The method of claim 77, wherein the subsequent sample was collected from the subject without assistance of a phlebotomist, nurse, physician or other licensed health care professional.

83. The method of claim 77, wherein monitoring the subsequent sample comprises high sensitivity tests on samples that were obtained from the subject at least one, two, or three weeks apart from each other.

84. The method of claim 77, further comprising confirmatory testing if the presence of CMV nucleic acid in the subsequent sample is detected.

85. The method of claim 84, wherein the confirmatory testing comprises detecting consecutive changes in quantitative CMV nucleic acid measurements or in CMV viral titer measurements.

86. The method of claim 77, wherein the high-sensitivity test is selected from the group consisting of polymerase chain reaction (PCR), reverse transcriptase (RT)-PCR, sequencing, rolling circle amplification, and isothermal amplification.

87. The method of claim 77, wherein the sample is selected from the group consisting of whole blood, serum, plasma, urine, and saliva.

88. A method of determining a presence of active primary cytomegalovirus (CMV) infection, the method comprising:
collecting a sample from a pregnant subject at a first stage;
receiving a determination of a presence or absence of anti-CMV IgG in the sample, said determination comprising an immunoassay test, wherein an absence of anti-CMV IgG indicates that the subject is at-risk for CMV infection;
if the subject is at-risk, receiving results of monitoring a subsequent sample of the subject obtained from the subject at a subsequent stage,
the subsequent sample comprising nucleic acid of the subject,
said monitoring comprising detecting a presence or absence of CMV nucleic acid sequences in the subsequent sample by a high-sensitivity test,
wherein an absence of anti-CMV IgG in the sample and a presence of CMV nucleic acid in the subsequent sample indicate a presence of active primary CMV infection.

89. A method of treating, ameliorating, or inhibiting cytomegalovirus (CMV) infection in a pregnant subject, the method comprising:
receiving a result of an immunoassay test to determine a presence or absence of anti-CMV IgG in a sample obtained from the pregnant subject;
if anti-CMV IgG is absent from the sample:
determining the subject to be at-risk for CMV infection; and
receiving results of a high sensitivity test to determine a presence or absence of CMV nucleic acid sequences in a subsequent sample obtained from the subject;
determining the subject to have a CMV infection when anti-CMV IgG is absent and CMV nucleic acid is present; and
administering a CMV therapy to the pregnant subject determined to have the CMV infection.

90. The method of any one of claim 89, wherein the subsequent sample was collected from the pregnant subject in the absence of any phlebotomist, nurse, physician or other licensed health care professional.

91. The method of claim 89, wherein determining the subject to have an active, primary CMV infection comprises receiving at least one of:
a measurement of total CMV nucleic acid in the subsequent sample of the subject, or
a measurement of a rate of change in CMV nucleic acid levels in the subject.

92. The method of claim 89, wherein the CMV therapy is administered to the pregnant subject during the first half of the pregnancy, within three weeks of infection, and/or prior to transmission of CMV to a fetus or appearance of CMV symptoms in the fetus.

93. The method of claim 89, wherein the CMV therapy is selected from the group consisting of an antiviral compound, a biologic, an immunotherapy, and termination of a pregnancy, or a combination of two or more of the listed items.

94. The method of claim 93, wherein the antiviral compound is selected from the group consisting of acyclovir, valacyclovir, ganciclovir, valganciclovir, foscarnet, cytogam and cidofovir, and two or more of any of the listed items.

95. The method of claim 89, wherein if the CMV nucleic acid sequences are detected, the method further comprises confirmatory testing.

96. The method of claim 89, further comprising monitoring subsequent samples of the subject after a start of the CMV therapy to detect changes in viral load or titer, wherein a decrease in viral load or titer after the start of the CMV therapy indicates a response to the CMV therapy.

97. The method of claim 89, wherein the sample is selected from the group consisting of whole blood, serum, plasma, urine, and saliva.
